(19) United States
(12) Patent Application Publication
(10) Pub. No.: US 2024/0180994 A1
(43) Pub. Date: Jun. 6, 2024

(54) HERBAL EXTRACT FORMULA FOR TREATING CORONAVIRUS INFECTION

(71) Applicant: WAHI PHARMACEUTICALS INC., Winter Park, FL (US)

(72) Inventors: Michael HAKKY, Winter Park, FL (US); Shahad AL-FARHOOD, Epsom (GB)

(21) Appl. No.: 18/549,870

(22) PCT Filed: Mar. 9, 2022
(86) PCT No.: PCT/US2022/019643

§ 371 Date: Sep. 8, 2023

Related U.S. Application Data
(60) Provisional application No. 63/200,485, filed on Mar. 10, 2021.

(51) Int. Cl. A61K 36/86; A61K 36/28; A61K 36/47; A61K 36/60; A61K 36/61; A61K 36/81; A61K 36/84; A61K 36/896; A61K 36/906; A61P 31/14

(52) U.S. Cl. CPC A61K 36/86; A61K 36/28; A61K 36/47; A61K 36/60; A61K 36/61; A61K 36/81; A61K 36/84; A61K 36/896; A61K 36/906; A61P 31/14

(57) ABSTRACT

A composition comprising (a) an extract of a plant belonging to the family Violaceae; (b) an extract of a plant belonging to the family Myrtaceae; and one or more additional plant extracts wherein the plants are selected from the Moraceae Zingiberaceae, Hyoscamus, Asteraceae, Euphorbiaceae, Iridaceae, and/or Valeriana families. The composition is useful for preventing or ameliorating the effects of infection by a coronavirus including SARS-CoV-2.

### CROSS-REFERENCE TO RELATED APPLICATION

This application claims the benefit of priority to U.S. Patent Application Ser. No. 63/200,485 filed Mar. 10, 2021, the entire contents of which are incorporated herein by reference.

### FIELD OF THE INVENTION

The invention is directed to compositions and methods to prevent and reduce coronavirus pathogenesis and symptoms.

### BACKGROUND OF THE INVENTION

Coronaviruses are a large family of viruses that are common in people and many different species of animals, including camels, cattle, cats, and bats. Rarely, animal coronaviruses can infect people and then spread between people such as with MERS-CoV, SARS-CoV, and now with a new virus (named SARS-CoV-2). The SARS-CoV-2 virus is a Betacoronavirus, like MERS-CoV and SARS-CoV. All three of these viruses appear to have their origins in bats.

In 2019, the new coronavirus, named severe acute respiratory syndrome coronavirus 2 (SARS-CoV-2), was identified as the cause of a disease termed COVID-19. The COVID-19 outbreak that originated in China caused fear in the whole world. This worldwide fear is because of its fast-spreading infection. Initial symptoms of this disease caused by this virus include cough (this can be any kind of cough, but usually dry cough), shortness of breath and other breathing difficulties, fatigue, aches and pains, headaches, diarrhea (rare), and runny nose. The clinical course of the disease is widely variable and unpredictable, ranging from asymptomatic infection to multi-organ system failure, mainly lungs and kidneys, and death.

Through the end of 2021 over 300 million cases and 5.5 million deaths have been reported worldwide due to COVID-19. Although several vaccines against the virus are available, many people are reluctant to get vaccinated, leading to more infections and the chances of new mutants arise. Such mutants are more infectious and spread faster. Furthermore, vaccine efficacy is not 100% and immunity to new variants is often suboptimal even in fully vaccinated individuals. In addition, people are still vulnerable to infection following vaccination and before developing a complete immune response. Due to these factors, vaccination does not represent the definitive therapeutic to eliminate COVID-19 and the need for new therapeutic measures is ultimately warranted.

SARS-CoV-2 is the newest member of a large group of viruses coming under the order Nidovirales and family Coronaviridae and genera beta coronavirus (group 2B).

Human coronaviruses (HCoVs) include severe acute respiratory syndrome coronavirus (SARS-CoV) and 2019 novel coronavirus (2019-nCoV, also known as SARS-CoV-2). Phylogenetic analyses of 15 HCoV whole genomes reveal that 2019-nCoV/SARS-CoV-2 shares the highest nucleotide sequence identity with SARS-CoV (79.7%). Specifically, the envelope and nucleocapsid proteins of 2019-nCoV/SARS-CoV-2 are two evolutionarily conserved regions, having sequence identities of 96% and 89.6%, respectively, compared to SARS-CoV.

SARS-CoV-2 comprises four basic structural proteins, which are a club-shaped trimeric “spike protein (S)”, a “membrane (M) protein”, an “envelope (E) protein,” and a “nucleocapsid protein (N).” The infection process starts with the binding of the spike protein S1 receptor binding domain to the human host cell receptor angiotensin-converting enzyme 2 (ACE2), which leads to conformational change in the S1 and S2 domains of the spike protein. The epithelial cells of the lungs express transmembrane protease serine 2 (TMPRSS2) enzyme that cuts open the spike protein of the virus, exposing a fusion peptide in the S2 subunit, which mediates the fusion of the viral and host cell membranes. The latter creates an endosome inside the cell's cytoplasm, and with the effect of host cathepsin, a serine protease cleaves the endosome, and the RNA is released to the ribosomes/endoplasmic reticulum. Since the SARS-CoV-2 is a +ssRNA virus, the +ve strand uses the cellular ribosomes to be translated to viral proteins followed by its replication. During viral translation process, the virus uses its own protease, 3CL or Mpro, which cleaves the translated polyproteins. This process is essential for the SARS-CoV-2 replication. Furthermore, the assembly of the virus and the virus release is complex. The former depends on M, E, and N proteins for envelop formation, and virion assembly, whereas the viral release depends on the ion channels.

It is desirable to find compositions useful for treating subjects exposed to SARS-CoV-2 to prevent infection and/or reduce symptoms of COVID-19 caused by infection by SARS-CoV-2.

### SUMMARY OF THE INVENTION

This invention relates to natural compositions for the inhibition, prevention and/or treatment of infection with coronaviruses, including SARS-CoV-2, the causative agent of COVID-19. In some embodiments, the natural compositions include one or more botanical compounds dissolved in a specific manner to increase their bioavailability, while maintaining stability.

In one aspect, the composition comprises (a) an extract of a plant belonging to the family Violaceae; (b) an extract of a plant belonging to the family Myrtaceae; and one or more additional plant extracts wherein the plants are selected from the Moraceae Zingiberaceae, Hyoscamus, Asteraceae, Euphorbiaceae, Iridaceae, or Valeriana families.

In one embodiment, the composition comprises (a) an extract of a plant belonging to the family Violaceae; (b) an extract of a plant belonging to the family Myrtaceae; and (c) an extract of a plant belonging to the family Moraceae.

In some embodiments, the composition further comprises (d) an extract of a plant belonging to the family Iridaceae; and either (e) an extract of a plant belonging to the family Asteraceae; or (f) an extract of a plant belonging to the family Zingiberaceae.

The plant belonging to the family Violaceae includes but is not limited to Viola odorata, the plant belonging to the family Myrtaceae includes but is not limited to Leptospermum scoparium, the plant belonging to the family Moraceae includes but is not limited to Ficus carica, the plant belonging to the family Iridaceae includes but is not limited to Crocus sativus, the plant belonging to the family Asteraceae includes but is not limited to Anacyclus pyrethrum, and the plant belonging to the family Zingiberaceae includes but is not limited to Elettaria cardamomum.

In one embodiment, the composition comprises 20 to 25% of (a); 20 to 30% of (b); 20 to 40% of (c); 10 to 25% of (d); and 10 to 25% of (e); wherein the percentages of (a), (b), (c), (d) and (e) total 100%.

In another embodiment, the composition comprises 20 to 25% of (a); 20 to 25% of (b); 20 to 25% of (c); 20 to 25% of (d); and 10 to 15% of (e).

In yet another embodiment, the composition comprises 20 to 25% of (a); 20 to 30% of (b); 20 to 40% of (c); 10 to 25% of (d); and 10 to 25% of (f); wherein the percentages of (a), (b), (c), (d) and (f) total 100%.

In yet another embodiment, the composition comprises 20 to 25% of (a); 20 to 25% of (b); 20 to 25% of (c); 15 to 20% of (d); and 15 to 20% of (f).

In yet another embodiment, the composition comprises extracts of plants belonging to the Violaceae, Mvrtaceae, Zingiberaceae, Asteraceae, Iridaceae, and Valeriana families.

The compositions may further comprise one or more additional components selected from the group consisting of a carrier, an excipient, a binder, a colorant, a flavoring agent, a preservative, a buffer, a diluent, and any combination thereof.

The compositions embodied herein may be in various dosage forms, including but not limited to, a capsule, a cachet, a pill, a tablet, a powder, a granule, a pellet, a bead, a particle, a troche, a lozenge, and a gel.

The composition may be a dietary supplement, such as an over-the-counter product. In other embodiments, the composition may be a pharmaceutical composition, such as a prescription drug.

In another aspect, the invention provides a composition useful for treating an individual exposed to coronavirus and a method of treating the individual exposed to coronavirus or infected with coronavirus. The coronavirus may be but is not limited to SARS-CoV-2 or a variant thereof.

It has been found that the composition exhibits suppression of a viral protease. For example, the viral protease is 3CL (Mpro) of SARS-CoV-2.

It has been found that the composition exhibits inhibition of coronavirus S1+S2 spike proteins binding to ACE2 in the individual exposed to the coronavirus.

It has been found that the composition inhibits replication of the coronavirus in the individual exposed to the coronavirus.

The composition also exhibits inhibition of transmembrane protease serine 2 enzyme of lung epithelial cells in the individual.

The composition may inhibit a cytokine storm in an individual.

In another aspect of the invention, a method for treating coronavirus, such as SARS-CoV-2 or variants thereof, in an individual is provided. The method comprises administering a therapeutically effective amount of a composition as described above, including any of the embodiments.

The method reduces virus infectivity and pathogenic effects in the individual.

The method described herein suppresses viral protease, such as 3CL (Mpro) of SARS-CoV-2.

Thus, the method inhibits replication of the coronavirus in the individual exposed to the coronavirus.

The method also exhibits inhibition of coronavirus S1+S2 spike proteins binding to ACE2 in the individual exposed to the coronavirus.

The method wherein the composition exhibits inhibition of transmembrane protease serine 2 enzyme of lung epithelial cells in the individual.

The method wherein the composition inhibits a cytokine storm in an individual.

The method wherein the composition ameliorates coronavirus infection in the infected individual's lungs, kidneys and other body systems.

The method wherein the composition provides support for reducing systemic inflammation, and inflammation in the kidneys and lungs.

The method including the step of administering said composition orally.

Also provided is a method of preventing infection of an individual exposed to a coronavirus comprising administering a therapeutically effective amount of a composition as described above, including any of the embodiments alone or in any combination, wherein the composition reduces virus infectivity.

In embodiments of the method, the composition reduces virus infectivity in the individual, including infectivity of coronaviruses such as SARS-CoV-2 or variants thereof.

Administration of the composition may suppress a viral protease, such as 3CL (Mpro) of SARS-CoV-2.

Administration of the composition may inhibit replication of the coronavirus in the individual exposed to the coronavirus. The composition may inhibit coronavirus S1+S2 spike proteins binding to ACE2 in the individual exposed to the coronavirus. The composition may exhibit inhibition of transmembrane protease serine 2 enzyme of lung epithelial cells in the individual.

Administration of the composition may inhibit a cytokine storm resulting from infection by the SARS-CoV-2 virus.

The composition may be administered orally.

### BRIEF DESCRIPTION OF THE DRAWINGS

The disclosed aspects will be described in conjunction with the appended drawings, provided to illustrate and not to limit the disclosed aspects.

FIG. 1 shows aspects of inhibition of S1+S2 binding to ACE2 by each extract, according to embodiments of the invention.

FIG. 2 shows aspects of the inhibition of binding of S1+S2 to ACE2 by two extract combinations, according to embodiments of the invention.

FIG. 3 shows aspects of the inhibition of binding of S1 to ACE2 by two extract combinations, according to embodiments of the invention.

FIG. 4 shows a plot of the % spike inhibition of the two SARS-CoV-2 mutant variants B1.351 and B1.1.7, according to embodiments of the invention.

FIG. 5 shows aspects of the reduction of TMPRSS2 activity by different concentrations of two extract combinations, according to embodiments of the invention.

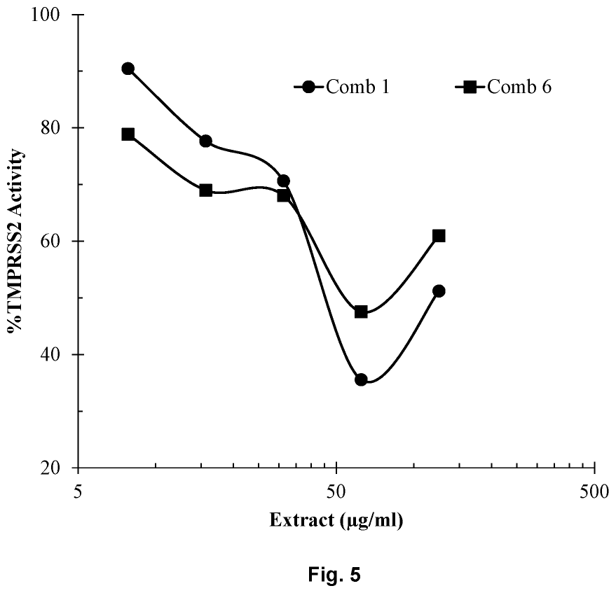

FIG. 6 shows aspects of the reduction of 3CL activity by different concentrations of two extract combinations, according to embodiments of the invention.

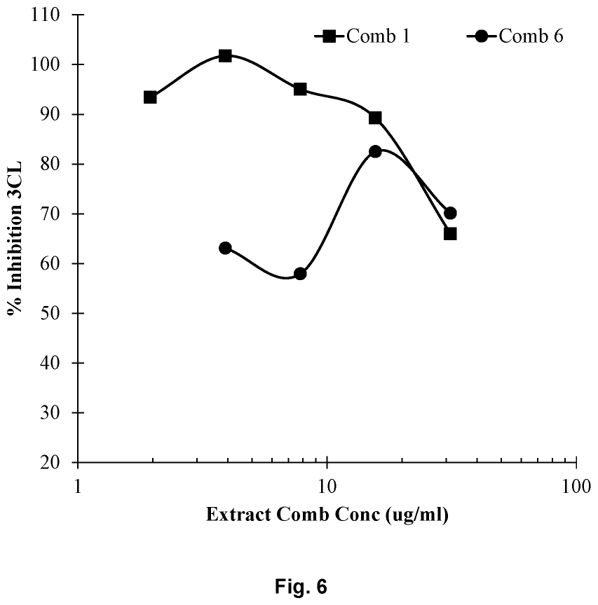

FIG. 7 shows a chart of body weight (g) of mice in a toxicity study during administration of test compositions according to embodiments of the invention.

FIG. 8 shows a chart of body weight change (%) of mice in a toxicity study during administration of test compositions according to embodiments of the invention.

FIG. 9 shows a dose response curve for cytotoxicity of Vero-E6 Cells by a test composition according to embodiments of the invention.

FIG. 10 shows a dose response curve for cytotoxicity of Vero-E6 Cells by another test composition according to embodiments of the invention.

FIG. 11 shows a chart of body weight change (%) in mice during administration of test compounds (Combo 1 and 10) for 7 days in comparison to placebo, and control.

FIG. 12 shows aspects of the effect of pre-administration of two extract combinations, according to embodiments of the invention, for seven days at 200 mg/kg/day on LPS-induced IL-6 production in blood, kidneys, and lungs.

FIG. 13 shows aspects of the effect of pre-administration of two extract combinations, according to embodiments of the invention, for seven days at 200 mg/kg/day on LPS-induced IL-1β production in blood, kidneys, and lungs.

In the following description, for purposes of explanation rather than limitation, specific details are set forth such as the compositions used and their benefits and side effects individually and when combined to provide a thorough understanding of the concepts of the invention. However, it will be apparent to those skilled in the art that this invention may be practiced in other embodiments, which depart from these specific details. In like manner, the text of this description is directed to the example embodiments as illustrated in the Figures and is not intended to limit the claimed invention beyond the limits expressly included in the claims.

### DETAILED DESCRIPTION OF THE DISCLOSED SUBJECT MATTER

In one aspect, the present invention provides a composition having antiviral activity against coronaviruses, such as SARS-CoV-2 or variants thereof. In some embodiments, the composition includes a combination of all-natural compounds that provides prophylaxis against SARS-CoV-2 infection, inhibits its proliferation and/or reduces the inflammatory process in an active infection. In one embodiment, the composition comprises (a) an extract of a plant belonging to the family Violaceae; (b) an extract of a plant belonging to the family Myrtaceae; and (c) an extract of a plant belonging to the family Moraceae. In embodiments, the composition preferably comprises (d) an extract of a plant belonging to the family Iridaceae; and either (e) an extract of a plant belonging to the family Asteraceae; or (f) an extract of a plant belonging to the family Zingiberaceae.

Unless expressly indicated otherwise, all percentages listed herein are weight percentages, based on weights (masses) of the listed components in a composition.

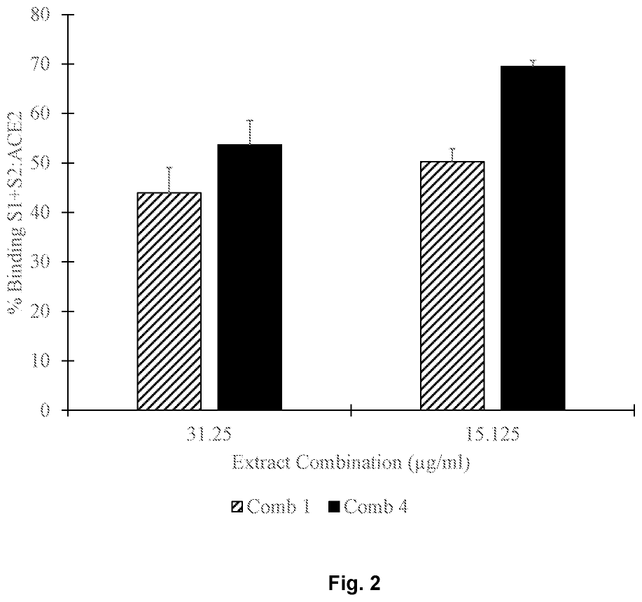

Coronavirus spike (S) glycoproteins promote the entry into cells and are the main target of antibodies. SARS-CoV-2 S protein uses the host ACE2 to enter cells, shown schematically in FIG. 2A. The receptor-binding domains of SARS-CoV-2 and SARS-CoV bind with similar affinities to human ACE2, correlating with the efficient spread of SARS-CoV-2 among humans.

Various clinically approved drugs that have been screened for their anti-SARS-CoV effects including nucleoside analogs, interferons, HIV protease inhibitors, antipsychotic drugs, anti-parasitic drugs and antibiotics.

As shown below, the compositions described herein inhibit the Spike protein on SARS-CoV and SARS-CoV-2 from binding to ACE2 receptors.

It has been found that the compositions described herein are effective in treating coronavirus, including COVID-19, or reducing the severity of symptoms or life of the infection. For example, the composition exhibits direct inhibition of spike (S1+S2) binding to ACE2 receptor and thereby reducing infectivity to human cells; inhibition of TMPRSS2 enzymatic activity and thereby inhibits viral fusion with the target cell and then entrance and infectivity to human cells; inhibition of SARS-CoV-2 3CL protease and host cell cathepsin L thereby inhibiting viral translation and replication; and/or reducing the production of pro-inflammatory cytokines and thereby reducing the inflammatory-induced damage inside the human body.

The composition is useful for treating SARS-CoV 2 virus pathogenesis. The constituents listed herein bind to the spike protein (S1+S2) of the SARS-CoV-2 virus and its mutants (variants) and the TMPRSS2 of the host and therefore inhibit viral infectivity to the human cells. Also, the constituents inhibit the coronavirus enzyme 3CL and therefore reduce viral replication inside the human cell. Further, the constituents reduce the SARS-CoV-2 inflammatory cytokine production. Thus, this invention should be useful for treating SARS-CoV-2 infection (COVID-19).

The composition includes (a) an extract of plants belonging to the family Violaceae, wherein said extract is obtainable or obtained by extracting at least aerial parts (flower and leaves) of the plant with hydrophilic, medium polar and/or lipophilic solvent. A notable plant in this family is Viola odorata (sweet violet). The aqueous preparations of Viola odorata L. flowering tops revealed the presence of anthocyanins. Furthermore, the compositions may optionally contain an alkaloid, glycoside, saponins, methyl salicylate, mucilage, cyclotide and vitamin C.

Viola odorata extracts can be used for ameliorating nervous strain, hysteria, physical and mental exhaustion, symptoms of menopause (hot flashes), depression, and irritability. They are also used for digestive tract complaints such as abdominal pain, swelling (inflammation) of the stomach and intestines and the tissues that line them, digestion problems caused by improper diet, gas, heartburn, gallbladder disorders, and loss of appetite. It has also been used for treatment of respiratory tract conditions, particularly dry or sore throat, stuffy nose, coughs, hoarseness and sudden (acute) and ongoing (chronic) bronchitis, asthma, emphysema, “dust-damaged” lungs, and swelling (inflammation) of the respiratory tract. Other uses include treating pain in the minor joints, fever, skin diseases, headache, trouble sleeping (insomnia), and tuberculosis. These herbal compositions are also used for involuntary urination (incontinence) in older people, bed-wetting, irritable bladder, and prostate conditions.

The aqueous preparations of Viola odorata L. flowering tops revealed the presence of anthocyanins. The analysis of essential oil composition of the leaves of Viola odorata L. revealed the presence of 25 identified compounds, representing 92.77% of the oil with butyl-2-ethyl hexyl phthalate (30.10%) and 5,6,7,7a-tetrahydro-4,4,7a-trimethyl-2(4H)-benzofuranone (12.03%) being the two main components. Viola odorata also produces macrocyclic peptides. About 30 cyclotide proteins are identified from the aerial parts and roots of Viola odorata. According to the HS-SPME GC/MS analyses, ethyl hexanoate and (2E,6Z)-nona-2,6-dienol were specific volatile compounds of sample with French origins, while (E, E)-hepta-2,4-dienal, hexanoic acid, limonene, tridecane are found in extracts from other locales.

In some embodiments, the composition employs the whole aerial part including stem, flowers, and leaves which are believed to produce pharmacological activities, such as:

Viola odorata is known for its anti-inflammatory and anti-pyretic activities even in neutropenic children during hospital course. The anti-inflammatory activity was evident in preventing lung damage, bronchitis and coughing conditions. Furthermore, LPS-treated mice administered with oral doses of Viola odorata for 21 days exhibited reduced TNF-α, NF-kB and cyclo-oxygenase. Viola Odorata extract has dose-dependent inhibitory effects. It is also appropriate to give the dose depending on several factors such as the user's age, health, and several other conditions. The flower infusion can be taken in a dose of 30-60 ml. The flowers may be taken in a dose of ½- to 5 grams. The root infusion may be taken in a dose of 15-30 ml twice a day. 5-6 leaves can be chewed. The roots and seeds are poisonous in large doses. Roots are emetic in large doses. The oil should not be used internally. It may cause photosensitization when applied externally. Safety during pregnancy and lactation has not been established. In the absence of sufficient data, the use during pregnancy and lactation is not recommended.

The composition includes (b) an extract of a plant belonging to the family Mfyrtaceae, wherein said extract is obtainable or obtained by extracting Manuka Honey with hydrophilic, medium polar and/or lipophilic solvent. A notable plant in this family is Leptospermum scoparium, commonly known as Manuka. Extracts of this plant are conveniently obtained from Manuka honey produced by bees feeding on Manuka flowers. This honey is made by bees that pollinate Manuka flower tree (Mystacese). Most commonly this is done by European Honeybees (Apis mellifera).

Manuka honey inhibits influenza virus replication and has the strongest anti-influenza viral activity among most honey types, which is indicative of its antiviral effects.

Manuka honey contains around seventy compounds including amino acids and xanthophylle derivatives, mannitol, dylsitol, methylglyoxal and some aldehydes and esters. Mineral salts such as those containing calcium, sodium, potassium, manganese, iron, aluminum, and zinc cations and/or chlorine anions also have been identified. Manuka honey components have been considered as antioxidants, anti-viral or immunomodulatory agents and have been used to treat hyperthermia, tonsillitis, respiratory lesions, and increasing the immunity of the patient. It also improves the functions of the kidneys.

Manuka honey extract was chosen for its main active constituents, which include phenolic acids and flavonoids such as luteolin, quercetin, kaempferol and hesperidin.

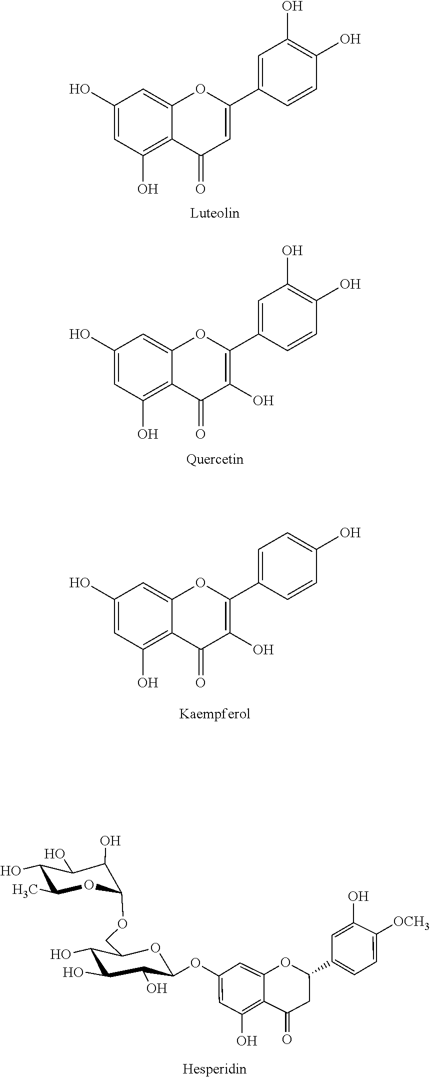

Luteolin has been found to inhibit entry of HIV-luc/SARS pseudotyped virus into host cells. Different concentrations of luteolin were added to the infection mixture. The effective dose at 50% of inhibition (EC50%) was 9.02 μM. Furthermore, luteolin was found to inhibit wild-type SARS-CoV cytopathic effect by using a MTT assay at EC50% of 10.6 μM. These values were 15 times less than the cytotoxicity of luteolin on the cells in vitro.

Quercetin has a similar structure to luteolin and has been determined by the US FDA as “Generally Recognized as Safe” and is expected to have an inhibitory activity against wild-type SARS-CoV. The EC50% of quercetin was 83.4 μM. Also, quercetin was found to inhibit cathepsin L and is considered as a natural cathepsin inhibitor.

Kaempferol has been found to inhibit SARS-CoV-2 protease 3CL pro at IC50% of 116.3 μM. Furthermore, kaempferol at concentrations at 62.5 to 125 μM protected SARS-CoV-2 cells infection of Vero cell line. These studies compliment the docking studies that showed that kaempferol inhibits the proteolytic activity of SARS-CoV 3CLpro. Furthermore, kaempferol glycoside at 10 μM inhibited the 3a ion channel of SARS-CoV2. This blocking may affect virus release from the infected cell.

As for hesperidin, several docking studies revealed that it has a potential inhibition activity against the SARS-CoV-2 exon. Hesperidin's agylcone is known as hesperitin. Furthermore, hesperidin exhibited lowest energy binding with docking scores of −13.51, −9.61, and −9.50 to the respective receptors of SARS-CoV-2 protease (6LU7), spike glycoprotein-RBD (6LXT), and PD-ACE2 (6VW1). The docking score of hesperidin to SARS-CoV-2 protease was less than lopinavir, the repurposing drug that is being conducted in clinical trials for COVID-19 treatment. This finding suggest that hesperidin achieves better interaction to the SARS-CoV-2 protease compared to lopinavir. Besides, hesperidin alleviated influenza-A induced lung injury, through inhibiting cytokine-induced damage. Also, hesperidin was found to decease TNF-α in mice treated with LPS.

The composition also includes (c) an extract of a plant belonging to the family 1Moraceae, wherein said extract is obtainable or obtained by extracting the fruit hydrophilic, medium polar and/or lipophilic solvent. Ficus carcia belongs to the family Moraceae. The fruit of this plant are the edible fruit known as figs. The fruit has fifteen anthocyanins, most of which contain cyanidin as aglycone and some pelargonidin derivatives. Total and individual phenolic compounds such as, phenolic acid, chlorogenic acid, flavones, and flavonols, have been isolated from fresh and dried fig skins of Ficus carica. The figs, however, contained higher amounts of phenolics than the pulp of fresh fruits, owing to the contribution of the dry skin. Quercetin rutinoside is the major phenolic compound. Phenolic acids, 3-O- and 5-O-caffeoylquinic acids, ferulic acid, quercetin-3-O-glucoside, quercetin-3-O-rutinoside, psoralen, and bergapten, and organic acids (oxalic, citric, malic, shikimic, and fumaric acids) have been isolated from the water extracts of the leaves of Ficus carcia. On the other hand, figs, the fruit, are rich in iron, calcium, copper, manganese, magnesium, and potassium. Also, the fruits contain gallic acid, chlorogenic acid, flavonoids, catechin, and rutin.

Of the Ficus constituents, other than quercetin (discussed earlier), rutin, caffeic acid and ferulic acid were all found to inhibit SARS-CoV-2 membrane envelop protein formation in modeling studies. In that study, the authors studied targeting the envelope (E), membrane (M) and nucleocapsid (N) protein as a potential target during viral assembly, viral structure, and pathogenesis. It was found that rutin possessed higher affinity with E protein, whereas caffeic acid and ferulic acid exhibited a higher affinity with the M protein. Thus, the identified compounds may act as constituents of agents effective against SARS-CoV-2 by inhibiting the envelope formation, virion assembly and viral pathogenesis.

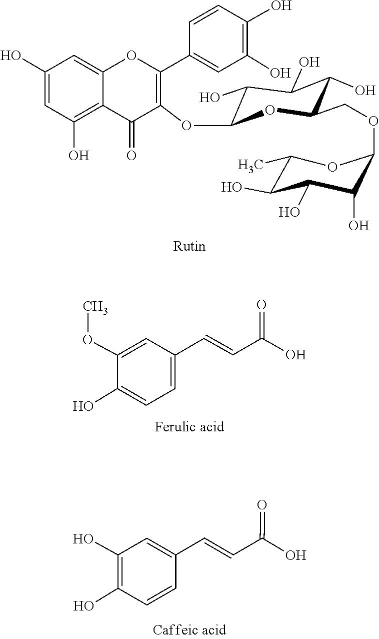

Optionally, the composition further comprises (d) an extract of a plant belonging to the family Iridaceae, wherein said extract is obtainable or obtained by extracting at least plant stigmas with hydrophilic, medium polar and/or lipophilic solvent. Crocus sativus (saffron crocus) belongs to the family Iridaceae, wherein said extract is obtainable or obtained by extracting at least plant stigmas with hydrophilic, medium polar and/or lipophilic solvent. This plant has about 150 volatile and non-volatile compounds. The volatiles include more than 34 components such as terpenes, terpene alcohols and their esters, whereas the non-volatile components, which are the active compounds in the composition described herein, include crocin, crocetin, picrocrocin and flavonoids, mainly quercetin, luteolin, hesperidin and kaempferol. Extracts are believed to improve asthma symptoms due to airway inflammation (anti-inflammation), hyper-responsiveness (immunomodulation) and muscle contraction (muscle relaxation) and have a bronchodilatory effect. Extracts have a positive effect on the cardiovascular system, CNS, gastrointestinal system, and respiratory system. They have also been used to treat skin, eye and infection diseases.

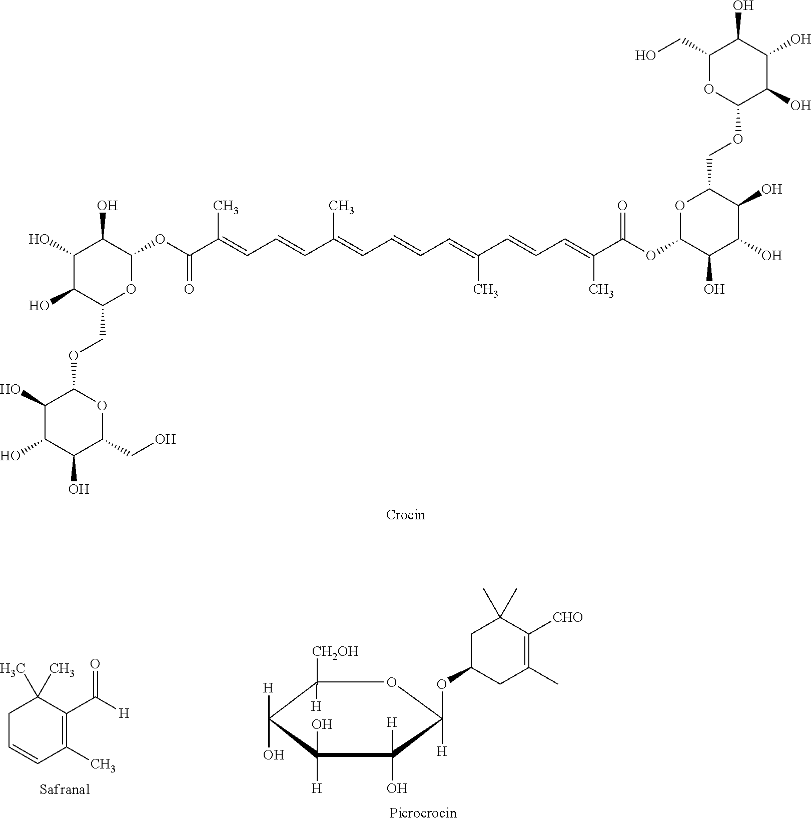

Safranal and crocin are the main components, along with crocetin (8,8′-Diapocarotenedioic acid), the agylcone of crocin. Both of these compounds were found to have anti-inflammatory effects. For instance, safranal (100 mg/kg) decreased the expression of the inflammatory cytokines TNF-α, IL-1β, and mitogen-activated protein kinases (MAPKs), such as the p38 in spinal cord injury models, but elevated the expression of the IL-1β, an anti-inflammatory cytokine, after spinal cord injury. Also, results showed that safranal could suppress the expression of aquaporin-4 (AQP-4), which is related to spinal-cord edema.

Saffron, especially its oil, facilitates breath and strengthens the respiratory organs especially in asthma patients. A potent stimulatory effect of C. sativus extract on beta2-adrenoceptors and histamine (H1) was also reported. This effect was due in part to its constituent, safranal.

Other flavonoids of saffron include crocin, a carotenoid pigment of saffron.

As for crocin, daily oral administration of crocin (10-30 mg/kg) for 4 weeks provides a protective effect not only on kidney organs by reducing the oxidative stress in aged rats but also significantly reduced pro-inflammatory cytokines, TNF-α, IL-6 and IL-1β, in the renal tissue and serum.

It has been found that crocin suppressed the LPS-induced activation of the MAPK pathway by inhibiting the phosphorylation of INK in lung tissues.

Optionally the composition comprises (e) an extract of plants belonging to the family Asteraceae, wherein said extract is obtainable or obtained by extracting the roots with hydrophilic, medium polar and/or lipophilic solvent. Anacyclus pyrethrum (Mount Atlas daisy) belongs to the family Asteraceae. The roots of the plant are rich in alkaloids or alkylamides mostly based on isobutylamide such as pellitorine or pyrethrine (N-isobutyldienediynamide), anacyclin, and other compounds including phenylethylamine, hydrocarolin, inulin, and sesamin. The antioxidant activity in vitro has revealed the presence of several antioxidant phytoconstituents such as flavonoids, alkamides, saponins and tannins in Anacyclus pyrethrum root extracts.

The water extraction of the plant exhibited immunostimulant activity.

Inulin, a polysaccharide, is the main ingredient for the immunostimulatory property of Anacyclus pyrethrum, whereas ether extraction revealed an immunomodulatory activity such as preserving phagocytosis, increasing antibody response, and increased delayed hypersensitivity response in the presence of cyclophosphamide).

Sesamin, on the other hand, has anti-inflammatory properties by modulating inflammation, T helper lymphocytes subtypes and therefore humoral and cellular immune responses. Furthermore, sesamin has a direct role in moderating the following signaling pathways: RAS/MAPK, PI3K/AKT, ERK1/2, p38, p53, IL-6, TNFα, and NF-κB signaling, which are all involved in inflammation, atherogenesis and hypertension.

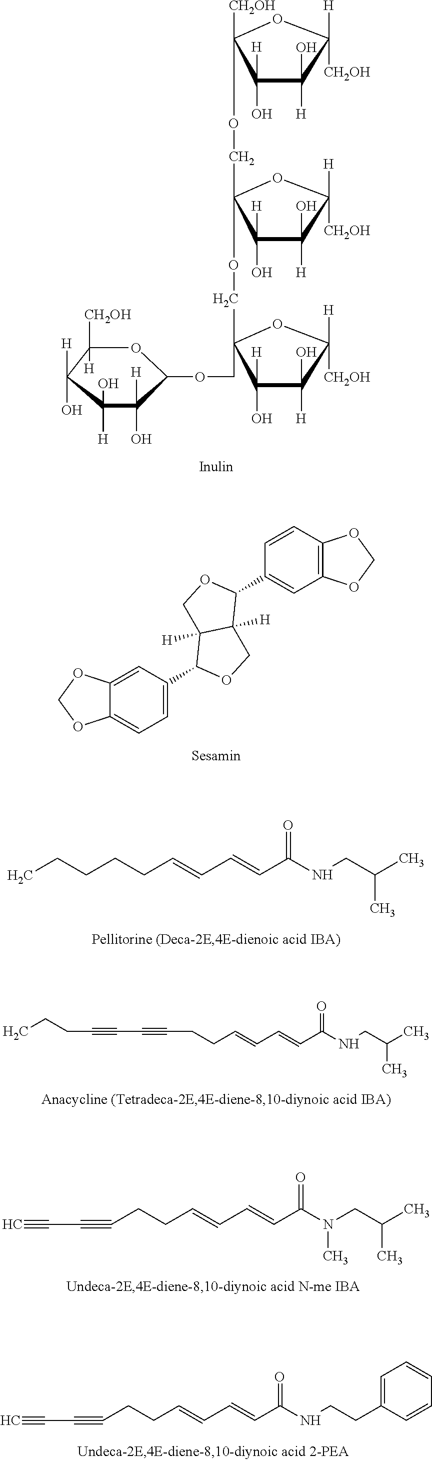

Optionally the composition comprises (f) an extract of a plant belonging to the family Zingiberaceae, such as Elettaria cardamomum (cardamom, sometimes cardamon or cardamum), wherein said extract is obtainable or obtained by extracting at least seeds and oil with hydrophilic, medium polar and/or lipophilic solvent. The oil extracted from cardamom seeds is a combination of terpene, esters, flavonoids (cardamonin, luteolin), phenolic compounds, tannins and saponins. The essential oil compounds could provide potential control of clinical pathogens. Cardamom essential oil showed diversity in chemical composition due to plant chemo types, climatic conditions, harvesting time and nutritional status. 1,8 Cineole and terpinyl acetate are the major active component of cardamom oil, which is a potent antiseptic. However, other constituents such as limonene, terpinolene and myrcene may add to its pharmacological activity along with the flavonoids in the fruit shell including cardamonin. Cardamonin was detected voltammetrically. Also, the dried seeds contain 1,8 cineole and terpinyl acetate which are the major active component of cardamom oil along with other constituents such as limonene, terpinolene and myrcene.

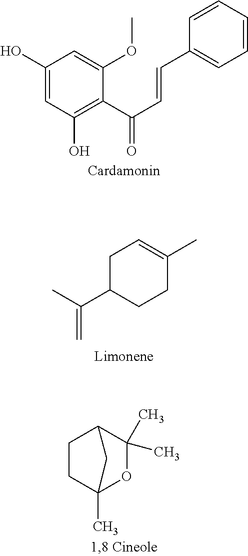

Cardomom oil is used primarily for its antifungal, antibacterial, antiviral, diuretic and carminative properties. It is also used against cardiac diseases, renal problems, anorexia, asthma and bronchitis. Because this treatment is taken orally, the antiseptic property may be helpful for the patients to reduce the risk of infection. Furthermore, it demonstrates antioxidant, anti-platelet aggregation, anti-hypersensitivity and anti-cancerous attributes.

Cardamonin is an immunomodulator that regulates a transcription factor, nuclear factor-KB (NF-κB), which enhances the transcription of proinflammatory cytokines). Also, it has been found that cardamonin protects against acute lung injury by regulating TLR2, 4-MyD88 and mTOR-autophagy pathways that are all associated with inflammatory-induced tissue injury. Furthermore, cardamonin can be a vasodilator by inhibiting the entry of calcium to the cell by the voltage-dependent Cav2.1 channel and stimulating the exit of potassium by the calcium-activated KCa1.1 channel.

Limonene is another compound that may have potential in preventing pulmonary fibrosis in COVID-19 patients. Limonene was found to inhibit PI3K/Akt/IKK-α/NF-κB p65 signaling pathway in COVID-19 pulmonary fibrosis. It has been demonstrated that the imbalance between collagen breakdown and metabolism, inflammatory response, and angiogenesis are the core processes of pulmonary fibrosis in SARS-CoV-2 infection, and PI3K/AKT signaling pathways are the key targets.

Another important molecule present in Elettaria cardamomum extracts is 1,8-cineole, which has known therapeutic benefits in inflammatory airway diseases, such as asthma and chronic obstructive pulmonary disease. The mechanism of action of 1,8-cineole includes controlling inflammatory processes and mucus hypersecretion.

We also investigated Valeriana wallichi (Valerian) extracts, which include alkaloids such as actinidine, chatinine, shyanthine, valerianine and valerene, isovaleramide, gamma-aminobutyric acid (GABA), Isovaleric acid; iridoids including valepotriates: isovaltrate and valtrate; sesquiterpenes (contained in the volatile oil) such as valerenic acid, hydroxyvalerenic acid and acetoxyvalerenic acid; flavanones: hesperidine, 6-methylapigenin and linarin where valeric is known for its anticonvulsant action. Extracts have been used for anxiety, insomnia and convulsion. Since the compounds in valerian produce CNS depression, desirably it is not used with other depressant drugs or antihistamines. There are no liver problems but there are some case studies in which hepatotoxicity has been observed in hypersensitive individuals following short-term use (i.e one month). Because of the uncertainty and the potential for toxicity in the fetus and hepatotoxicity in the mother, valerian use is discouraged during pregnancy. Extracts of sweet violet plant may be used in larger proportions in compositions comprising valerian to overcome these problems along with reducing gastrointestinal discomfort.

We also investigated extracts of Hyoscamus and Solanaceae families (henbanes). Coumarinolignans are major chemical constituents and yielded a new coumarinolignan, cleomiscosin A methyl ether along with four known coumarinolignans, cleomiscosin A, cleomiscosin B, cleomiscosin A-9′-acetate and cleomiscosin B-9′-acetate. The methanolic extract of seeds of H. niger (MHN) was evaluated for its analgesic, anti-inflammatory and antipyretic activities at different doses. It has an analgesic activity. Henbane is used in traditional herbal medicine for ailments of the bones, rheumatism, toothache, asthma, cough, nervous diseases, stomach pain and as a sedative.

Cleomiscosin A is an organic heterotricyclic compound; that is, 2,3-dihydro-9H-[1,4]dioxino[2,3-h]chromen-9-one substituted by a 4-hydroxy-3-methoxy phenyl group at position 3, a hydroxymethyl group at position 2 and a methoxy group at position 5 (the 2R,3R stereoisomer). It has a role as a metabolite and an anti-inflammatory agent.

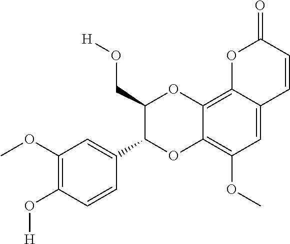 [SMILES: COc1cc([C@H]2Oc3c(OC)cc4ccc(=O)oc4c3O[C@@H]2CO)ccc1O]

Henbane ingestion by humans is followed simultaneously by peripheral inhibition and central stimulation, which gives the analgesic effect needed by the SARSCoV patients. This extract can be added in small amounts to reduce adverse effects, while experiencing its beneficial effects.

We also investigated extracts of Euphorbia umbellate (Euphorbiaceae). Euphorbia umbellata (E. umbellata) belongs to Euphorbiaceae family, popularly known as Janauba, and its latex contains a combination of phorbol esters with biological activities described to different cellular protein kinase C (PKC) isoforms.

Diterpenes are the major metabolites of these milk rich plants with many interesting structures, such as lathyranes, ingenanes, jatrophanes, tiglianes and myrsinols, in which some of them display various of biological activities including anti-HIV, cytotoxic, anti-inflammatory, antimicrobial, and modulation of multidrug resistance activities. Some ent-atisine diterpenes, ingol-type diterpenes, euphane and tirucallane triterpenoids were isolated from this plant in previous chemical research.

We also investigated extracts of Helleborus vesicarius (Ranunculaceae). Helleborus vesicarius grows in the border region between Syria and Turkey. Extracts include hellebrin, degluco-hellebrin, 20-hydroxyecdysone and protoanemonin, alkaloids, glycosides and saponosides. This class of natural compounds has a wide structural diversity, which may explain their multiple ranges of bioactivity reported so far.

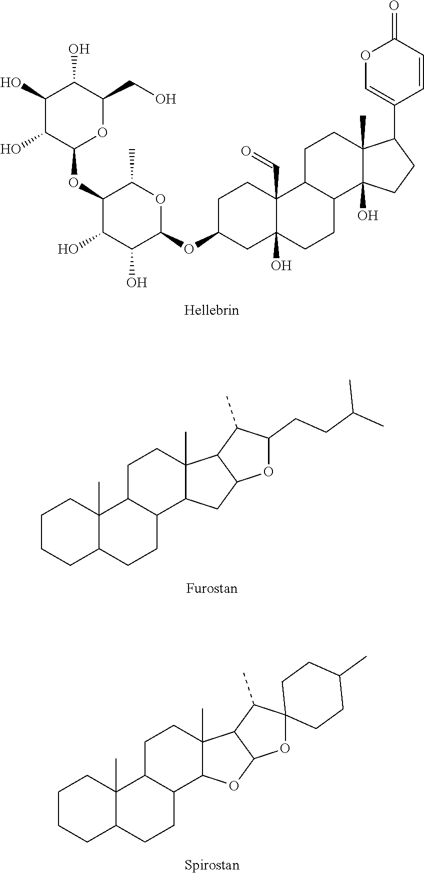 [SMILES: C[C@@H]1O[C@@H](O[C@H]2CC[C@]3(C=O)C4CC[C@]5(C)C(c6ccc(=O)oc6)CC[C@]5(O)C4CC[C@]3(O)C2)[C@H](O)[C@H](O)[C@H]1O[C@@H]1O[C@H](CO)[C@@H](O)[C@H](O)[C@H]1O ; CC(C)CCC1OC2CC3C4CCC5CCCCC5(C)C4CCC3(C)C2[C@@H]1C ; CC1CCC2(CC1)OC1OC3C4CCC5CCCCC5(C)C4CCC3(C)C1[C@@H]2C]

Some of the steroidal saponins (the aglycones are known as sapogenins) have cancer-related activity, as well as immunomodulating, antihepatotoxic, antiviral, and antifungal activities. They have been used for treatment of cancer, ulcers, diabetes and also for common medical problems such as toothache, eczema, low immunity and arthritis. They exhibit antihepatotoxic, antiviral, and antifungal activities.

This disclosure also discloses use of the compositions disclosed herein in treating SARS-CoV 2 virus pathogenesis. The constituents herein bind to the spike protein (S1+S2) and the TMPRSS2 of the SARS-CoV2 and its mutants and therefore inhibit viral infectivity to the human cells. Also, the constituents inhibit the enzyme 3CL and therefore reduce viral replication inside the human cell. Further, the constituents reduce the SARS-CoV-2 inflammatory cytokine production. Thus, this invention should be useful for treating SARS-CoV-2 infection (COVID-19).

### Spike Inhibition Tests

Many glycosylated spike proteins are expressed on the surface of SARS-CoV-2. This spike protein binds to the ACE2 receptor of the host cells, which facilitates viral entry inside the cells. As mentioned earlier, following the binding of the spike protein S1 to ACE2, a conformational change in the S1 and S2 domains occurs. The host epithelial cells express TMPRSS2 enzyme that cuts open the spike protein of the virus, exposing a fusion peptide in the S2 subunit, which mediates the fusion of the viral and host cell membranes. If S1 binding ACE2 is inhibited, viral entry will also be inhibited, and thus infection is prevented and reduced.

Initially, we tested each extract in inhibiting SARS spike (S1+S2) binding to ACE2 receptor in SARS-CoV-1 (SARS-CoV-1 Spike Trimer (S1+S2):ACE2 inhibitor screening colorimetric assay Kit (BPS Bioscience, Catalog #78012). SARS-CoV-1 spike protein is similar to SARS-CoV-2 and recognizes and binds to ACE2 receptor. Due to the similarities between the spike of SARS-CoV-1 and SARS-CoV-2, drugs that targets SARS-CoV-1 should also target SARS-CoV-2. However, it has been found the SARS-CoV-2 spike protein binds with higher affinity to ACE2.

The SARS-CoV-1 Spike Trimer (S1+S2):ACE2 Inhibitor Screening Assay Kit includes the SARS-CoV-1 Spike protein in its native trimeric conformation to provide a more physiologically relevant screen for inhibitors as well as a compatible platform to investigate the specificity of SARS-CoV-2:ACE2 inhibitors.

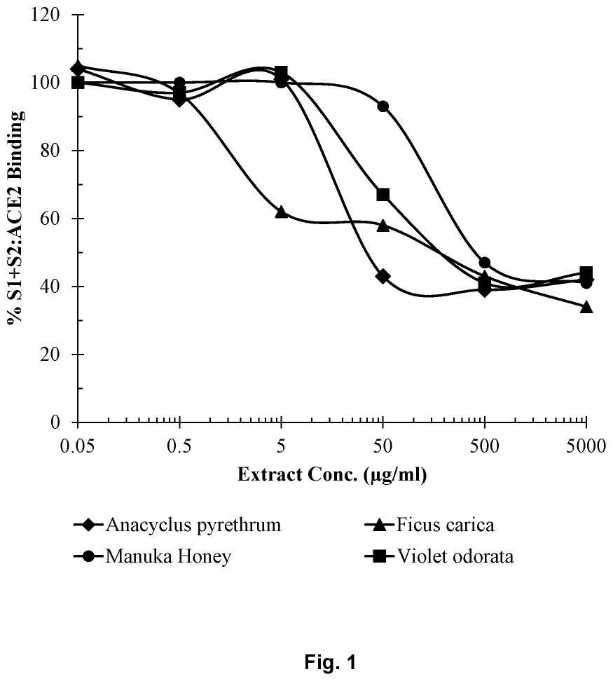

FIG. 1 shows a summary of inhibition of S1+S2 binding to ACE2 by extracts of Anacyclus pyrethrum, Ficus carica, Manuka Honey, and Violet odorata. All extracts inhibited S1+S2 binding to ACE2 receptor. The results showed that the concentrations at 30% inhibition and 50% inhibition of each extract were 19, 25, 200, and 47 μg/ml, and 30, 250, 400, 120 μg/ml for Anacyclus pyrethrum, Ficus carica, Manuka Honey, and Violet odorata, respectively.

### TMPRSS2 Inhibition

TMPRSS2 is a transmembrane protease serine 2 enzyme that is expressed on the epithelial cells of the host lungs. In SARS-CoV-2 infection and following SARS-CoV-2 binding to the ACE2 receptor, TMPRS22 primes the spike protein and cleaves at the S1/S2 cleavage site and opens the S2 subunit, which mediates the fusion of the viral and host cell membranes. Therefore, inhibiting or blocking the activity of TMPRS22 would halt SARS-CoV-2 entry into the cell and lowers viral infectivity.

To test whether the plant extracts can inhibit TMPRS22 activity, each of the ingredients above were tested using BPS Bioscience TMPRSS2 Fluorogenic assay kit (Cat #78083). Of the six extracts mentioned above, four (Anacyclus pyrethrum, Crocus sativus, Ficus carica, and Violet odorata) showed a mild to moderate degree (20-40%) of TMPRSS2 inhibition at concentrations of 2-200 μg/ml.

### 3CL Inhibition

When SARS-CoV-2 fuses with the target cell membrane, an endosome inside the cell's cytoplasm will form. A host cathepsin, which is a serine protease cleaves the endosome, and the RNA is released to the ribosomes/endoplasmic reticulum. Since the SARS-CoV-2 is a +ssRNA virus, the +ve strand uses the cellular ribosomes to be translated to viral proteins followed by its replication. During viral translation process, the virus uses its own protease, 3CL or Mpro, that cleaves the translated polyproteins. This process is essential for the SARS-CoV-2 replication. Thus, inhibiting 3CL enzyme will halt viral translation process and inhibits the production of new viral particles.

To test whether the plant extracts described herein can inhibit 3CL enzyme activity, each of the extracts above were tested using BPS Bioscience 3CL Protease, untagged SARS-CoV-2 Fluorogenic assay kit (Catalog #78042-1). Of the six extracts mentioned above, three (Anacyclus pyrethrum, Crocus sativus, and Manuka Honey) showed a moderate to high degree (40-85%) of 3CL inhibition at concentrations of 2-200 μg/ml.

Following single testing of proposed extracts on the spike, 3CL and TMPRS22 inhibitions, one proposed ingredient, Valeriana wallichi extract, did not show any meaningful effect on these tests. Therefore, it was removed from further testing.

We then tested different combinations and ratios of the extracts described herein and numbered each from 1 to 10 (Table 1). Since the efficacy of our compositions is on three SARS-CoV-2 mechanisms of action (spike, 3CL, and TMPRS22 inhibition), results from the three results were considered. All combinations proved to be effective with varying percentages of inhibition to the three mechanisms.

TABLE 1

| | Violet | | | Crocus | | |
|---|---|---|---|---|---|---|
| | odorata, | | | sativus, | | |
| | Violaceae | Myrtaceae | Ficus | Iridaceae | Anacyclus | Elettaria |
| | (Sweet | (Manuka | carica, | (Saffron | pyrethrum | cardamomum |
| | violet) | Honey) | (Moraceae) | crocus) | (Asteraceae) | (Cardamon) |
| Combination | Percentage | | | | | |
| 1 | 22.22 | 22.22 | 22.22 | 11.11 | 22.20 | 0 |
| 2 | 25 | 25 | 25 | 0 | 25 | 0 |
| 3 | 20 | 30 | 40 | 0 | 10 | 0 |
| 4 | 22.22 | 22.22 | 22.22 | 0 | 22.22 | 11.11 |
| 5 | 20 | 20 | 20 | 10 | 10 | 0 |
| 6 | 20 | 20 | 20 | 0 | 10 | 10 |
| 7 | 25 | 25 | 25 | 25 | 0 | 0 |
| 8 | 25 | 25 | 25 | 0 | 0 | 25 |
| 9 | 25 | 25 | 25 | 12.50 | 0 | 12.5 |
| 10 | 22.22 | 22.22 | 22.22 | 16.67 | 0 | 16.67 |

For example, two combinations, Combination 1 and Combination 4 were tested. Both Combination 1 and 4 have similar extracts (Anacyclus pyrethrum, Ficus carica, Manuka Honey, and Violet odorata) at 22.2% ratios except that Combination 1 contained Crocus sativus (11.1%), whereas Combination 4 contained Elettaria cardamomun (11.1%). FIG. 2 shows a bar graph of the inhibition of binding of S1+S2 to ACE2 at two different dosage rates. Both combinations inhibited the binding of S1+S2 to ACE2. However, the inhibition at lower concentration was more evident for Comb 1.

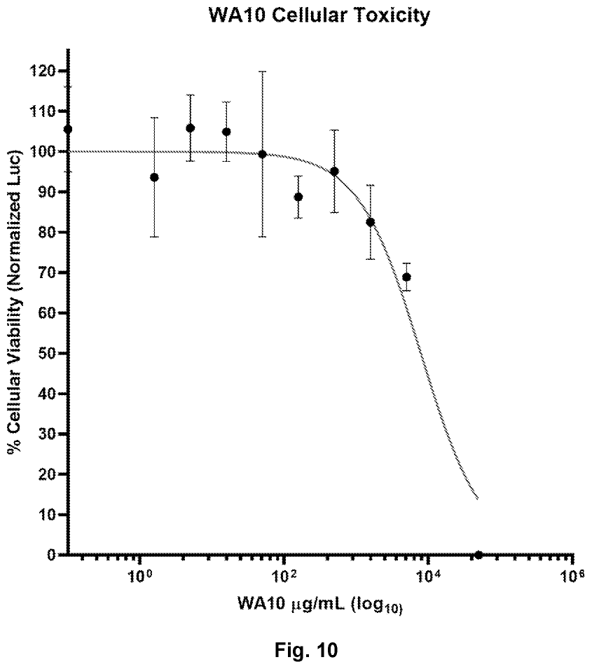

We tested Combination 1 and Combination 10 on SARS-CoV-2 spike binding to ACE2 (Cayman Chemical Cat. 502050). Both combinations contain Viola odorata, Manuka Honey, Ficus carica, and Crocus sativus. The difference in the two combinations is two-fold. Firstly, Combination 1 contained Anacyclus pyrethrum, whereas Combination 10 contained Elettaria cardamomum. Secondly, the ratios of the two constituents were different. Combination 1 contained 22.2% and 11.1% of Anacyclus pyrethrum and Crocus sativus, respectively, whereas Combination 10 contained 16.7% of both Crocus sativus and Elettaria cardamomum. The other three ingredients (22.2%) were similar in both combinations. The assay revealed that both combinations inhibited binding of S1 to ACE2, but Combination 1 showed significantly better inhibition activity (FIG. 10).

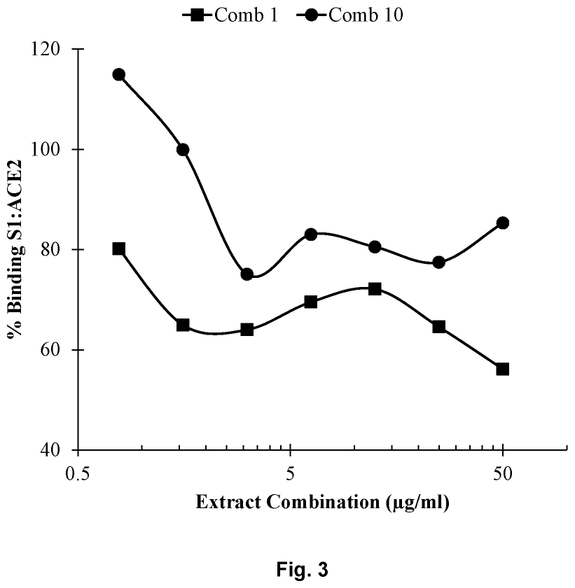

Lastly, we tested Combinations 1 and 10 against two mutant forms (B.1.1.7 and B.1.351 variants) using BPS Bioscience (Cat 78175 and 78171). Both combinations inhibited spike (S1+S2) binding to ACE2 receptor in a similar fashion (FIG. 3).

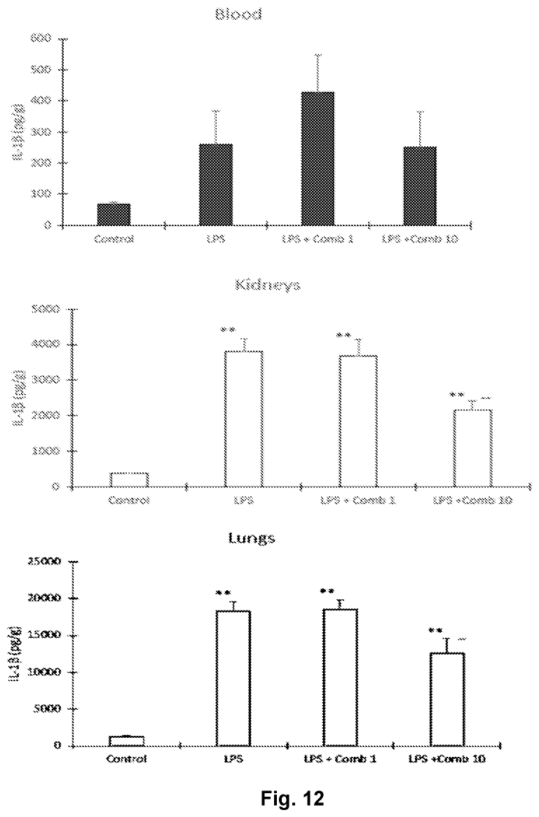

When several combinations were formulated and tested against TMPRSS2, the best inhibition was for Combination 1 and values ranged from 22-65% at 15.6-62.5 μg/ml, whereas for Combination 6 inhibition values ranged from 21-52% at 7.8-62.5 μg/ml (FIG. 12). Combination 1 included Anacyclus pyrethrum (22.2%), Crocus sativus (11.10%), Ficus carica (22.2%), Violet odorata (22.2%) and Manuka Honey (22.2%), whereas Combination 6 included Anacyclus pyrethrum (10%), Elettaria cardamomum (10%), Ficus carica (40%), Violet odorata (20%) and Manuka Honey (20%). These results indicate that combining the extracts has an additive effect on inhibiting TMPRSS2 activity.

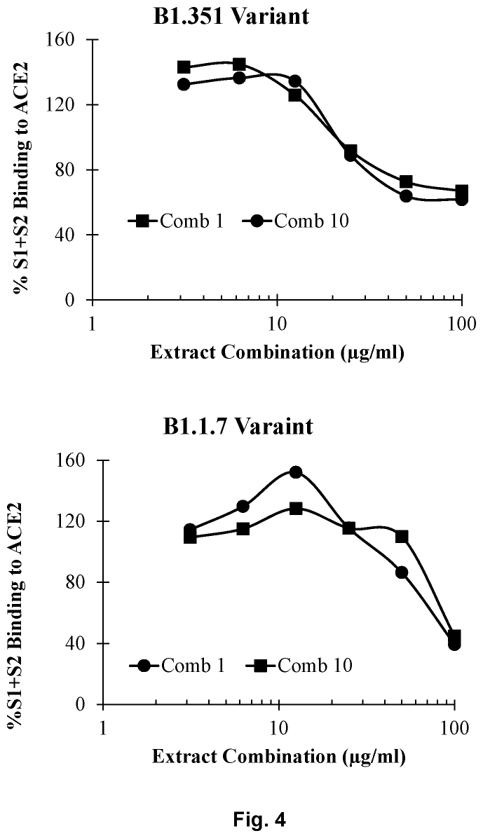

When several combinations were formulated and tested against 3CL, combination 1 showed 3CL inhibition ranging from 11-34% at 15.6-31.3 μg/ml, whereas combination 6 showed 3CL inhibition ranging from 19-40% at 3.9-15.6 μg/ml inhibited 3CL (FIG. 4).

Following testing of combinations on direct inhibition of spike (S1+S2) binding to ACE2, inhibition of TMPRSS2 enzymatic activity, and inhibition of SARS-CoV-2 3CL protease and considering all three tests, the best overall results were observed with Combinations 1 and 10.

Accordingly, two these combinations (Combinations 1 and 10) were set for testing cytokine modulation in vivo as described below. The difference in the two combinations is one ingredient. Both combinations contained Viola odorata, Manuka honey, Ficus carica, and Crocus sativus. The difference in the two combinations is two-fold. Firstly, Combination 1 contained Anacyclus pyrethrum, whereas Combination 10 contained Elettaria cardamomum. Secondly, the ratios of the two constituents were different. Combination 1 contained 22.2% and 11.1% of Anacyclus pyrethrum and Crocus sativus, respectively, whereas Combination 10 contained 16.7% of both Crocus sativus and Elettaria cardamomum. The other three ingredients (22.2%) were similar in both combinations.

To characterize the Combinations 1 and 10 for their usefulness in treating COVID-19, we carried out three additional tests. First, we tested cytokine response in an animal model of inflammation and cytokine storm. We also tested for acute toxicity and cytotoxicity.

### Cytokine Storm Modulation

Clinical data have shown that severe COVID-19 infections are associated with increased levels of inflammatory cytokines and chemokines. One of these increased inflammatory cytokines is IL-6, and it was found that increased blood IL-6 level is highly correlated with disease mortality. It has been shown that following SARS-CoV-2 binding to ACE-2 receptor enabling the endocytosis of ACE-2 and SARS-CoV-2 inside the infected cell. Such internalization of ACE-2 receptor reduces the ability of Ang II degradation and thus increases serum angiotensin II (Ang II) level. Ang II acts as a vasoconstrictor and it binds to AT1 receptor (AT1R) and activates downstream pathways such as MAPK pathway via ERK/JNK/p38, activation of JAK/STAT pathway via gp130, direct activation of NF-κB via p65. Such activation results in the production of inflammatory cytokines such as TNF-α, IL-1β, and many others, as well as IL-6 amplifier as positive feedback for inflammation non-immune cells as well as in immune cells. Thus, the cytokine storm caused by the hyperactivation of NF-κβ in the IL-6 amplifier may cause fatal symptoms such as coagulopathy, acute respiratory distress syndrome, severe pneumonia, and acute kidney injury in hospitalized COVID-19 patients. Thus, inhibiting or regulating IL-6 production could be a strategic treatment of severe COVID-19 infection.

### Toxicity Study

### Objective of the Study

This study was designed to test toxicity of combinations described herein. Objectives were to provide preliminary identification of target organs of toxicity, and occasionally, reveal delayed toxicity (if applicable); to determine the Median Lethal Dose (LD₅₀); to choose doses for repeat-dose studies; to determine reversibility of toxicity; and to identify parameters for clinical monitoring.

Seven test articles (TAs) in a powder form were used for the study. Combinations of the extracts were prepared as described in Table 2. The solubility of TAs in aqueous solution is acceptable.

All Test Articles (TAs) labelled in Table 2, containing Combinations 1, 4, 5 and 6, were stored in their original package as received. All preparations were freshly dissolved in 0.5% medium viscosity carboxymethyl cellulose (CMC) (Sigma-Aldrich, Missouri, USA) directly before experimentation or dosing and were discarded after use. All preparations were inspected visually for consistency and were shaken before use.

TABLE 2
Constituents of Employed Combinations (TAs)

| | Comb. 1 | Comb. 4 | Comb. 5 | Comb. 6 |
|---|---|---|---|---|
| TA001 | + | + | + | + |
| TA002 | + | − | + | − |
| TA003 | − | + | − | + |
| TA004 | + | + | + | + |
| TA005 | + | + | + | + |
| TA006 | − | − | − | − |
| TA007 | + | + | + | + |

### Selection and Assignment of Animals

Healthy non-pregnant mice were randomly selected, marked for individual identification, and kept in their cages for 8 days prior to the initiation of the study to allow acclimatization.

### Housing

Study groups were assigned into polysulfone individually ventilated cages (IVC) system; Eurostandard Type II H 1285L (floor area 542 cm², with wired inner fitting lid) (Techniplast, Italy). Mice were randomly assigned and housed with no more than 5 per cage, under controlled environmental conditions.

### Environmental Conditions

The animal housing room maintains a temperature of 22° C. (±3° C.) and 65% (±5) relative humidity and artificial photoperiodic lighting of 12 hr-light and 12 hr-dark cycle.

### Food

Mice were provided with standard rodent chow diet offered ad libitum. It is considered that there are no known contaminants in the feed that could interfere with the objectives of the study. Prior the administration of doses, animals were fasted for 4 hours from food.

### Water

Mice were provided with clean drinking water ad libitum. It is considered that there are no known contaminants in the water that could interfere with the objectives of the study. Throughout the study (even under food fasting conditions) animals were provided with water ad libitum.

### Methods

### Route of Administration

The TAs were delivered in vivo in a single dose by oral gavage using an oral gavage stainless steel needle of suitable size.

### Experimental Design and Justification of the Doses

### Sighting Study (Limit Test)

The purpose of the sighting study is to allow selection of the appropriate starting dose for the main study. The test substances were administered to single animals.

The doses for the sighting study were selected from the fixed dose levels of 5, 50, 300 and 2000 mg/kg, as recommended by the OECD Guideline for Testing of Chemicals (OECD, 420).

The highest dose for the sighting study was 2000 mg/kg, since the TAs were claimed safe by the sponsor and had no previous literature stating otherwise. Animals were observed closely for the first 24 hours, and kept for observation for 14 days.

### Cage-Side Observations

Cage-side observations of all groups are presented in Table 3. Parameters considered included response to external stimuli, grooming, general activity and movement. All animals were considered normal and did not expressed any abnormal behaviors or signs. No compound-related mortality or signs of toxicity were noted.

TABLE 3
Summary of Sighting Study Cage-side and Mortality Observation

| | Doses Employed for Limit test | | | | |
|---|---|---|---|---|---|
| | 5 mg/kg | 50 mg/kg | 300 mg/kg | 2000 mg/kg | Treatment |
| Number of Animals Observed | 1 | 1 | 1 | 1 | Combo 1 |
| No abnormalities detected | 1 | 1 | 1 | 1 | |
| Number of Animals Observed | 1 | 1 | 1 | 1 | Combo 4 |
| No abnormalities detected | 1 | 1 | 1 | 1 | |
| Number of Animals Observed | 1 | 1 | 1 | 1 | Combo 5 |
| No abnormalities detected | 1 | 1 | 1 | 1 | |
| Number of Animals Observed | 1 | 1 | 1 | 1 | |
| No abnormalities detected | 1 | 1 | 1 | 1 | Combo 6 |
| Mortalities | 0 | 0 | 0 | 0 | |

### Main Study

After determining the appropriate starting dose level from the limit test (sighting), specifically 2000 mg/kg, a total of five female mice were used to investigate the effect of the TAs (Combs. 1, 4, 5 and 6). Animals were observed closely for the first 24 hours, and kept for observation for 14 days.

### Cage-Side Observations

Cage-side observations of all groups are presented in Table 4. Parameters considered included response to external stimuli, grooming, general activity and movement. During the first hour from treatment, mice treated with Comb. 1 and Comb. 4 showed signs of calmness and less response to external stimuli. Such observation decline at the second hour from treatment.

TABLE 4
Summary of Main Study Cage-side and Mortality Observation

| | Comb. 1 | Comb. 4 | Comb. 5 | Comb. 6 |
|---|---|---|---|---|
| | (2000 | (2000 | (2000 | (2000 |
| Parameters Considered | mg/kg) | mg/kg) | mg/kg) | mg/kg) |
| No abnormalities | 5 | 5 | 5 | 5 |
| detected | | | | |
| Abnormality detected | 0 | 0 | 0 | 0 |
| Response to External | 0 | 0 | 0 | 0 |
| Stimuli | | | | |
| General activity (less) | 5* | 5* | 0 | 0 |
| Grooming | 0 | 0 | 0 | 0 |
| Sluggish Movement | 0 | 0 | 0 | 0 |

*less activity was noted only during the first hour from dosing

### Detailed Clinical Observations

A summary of detailed clinical observations are presented in Table 5. Parameters considered included passiveness to capturing, skin texture, respiratory sounds, abnormal discharges, and abnormal abdominal or urogenital presentation. All animals were handled and observed. Animals of groups treated with Comb. 1 and Comb. 4 showed normal response to capture and were comparably similar to other groups, thus, concluded normal with no clinically significant changes. No treatment-related observations were noted.

TABLE 5
Summary of Detailed Clinical Observations

| | Comb. 1 | Comb. 4 | Comb. 5 | Comb. 6 |
|---|---|---|---|---|
| | (2000 | (2000 | (2000 | (2000 |
| | mg/kg) | mg/kg) | mg/kg) | mg/kg) |
| Number of Animals | 5 | 5 | 5 | 5 |
| Observed | | | | |
| No abnormalities | 5 | 5 | 5 | 5 |
| detected | | | | |
| Abnormality detected | 0 | 0 | 0 | 0 |

### Body Weights and Body Weight Changes

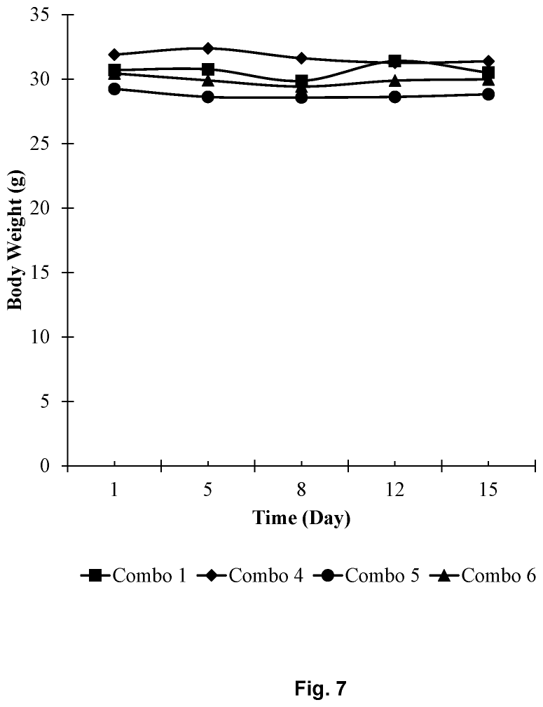

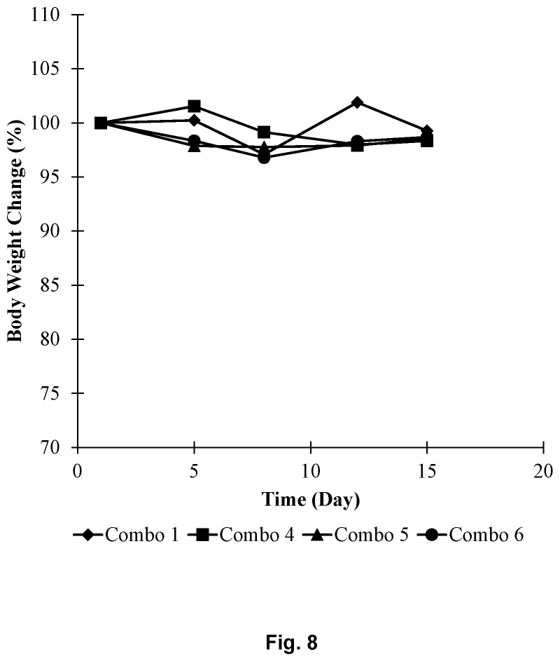

Group summary and individual body weight and body weight change data are presented in FIGS. 7 and 8 and Table 6. No compound-related body weight changes were noted. No significant differences were noted in absolute body weight or total body weight changes over the course of the study.

TABLE 6
Individual weights of animals (g)

| Combination | | (Day −2) | (Day 1) | | | Day 12 |
|---|---|---|---|---|---|---|
| (2000 mg/kg) | | Predosing | Dosing | Day 4 | Day 9 | (Terminal) |
| 1 | 1 | 28.0 | 28.3 | 27.1 | 27.1 | 27.5 |
| | 2 | 30.0 | 30.6 | 28.2 | 28.9 | 30.4 |
| | 3 | 33.3 | 32.7 | 33.0 | 37.1 | 32.9 |
| | 4 | 27.9 | 27.8 | 26.5 | 27.9 | 27.1 |
| | 5 | 34.3 | 34.4 | 34.5 | 36.0 | 34.6 |
| 4 | 1 | 31.7 | 32.0 | 31.7 | 31.5 | 30.6 |
| | 2 | 29.2 | 29.6 | 28.5 | 28.3 | 28.4 |
| | 3 | 31.3 | 32.9 | 32.0 | 30.9 | 31.4 |
| | 4 | 33.6 | 33.6 | 32.9 | 32.5 | 33.3 |
| | 5 | 33.7 | 33.8 | 33.0 | 33.1 | 33.2 |
| 5 | 1 | 27.2 | 26.7 | 26.8 | 27.1 | 26.6 |
| | 2 | 30.2 | 29.6 | 28.9 | 28.9 | 28.9 |
| | 3 | 29.8 | 28.7 | 28.6 | 29.1 | 29.1 |
| | 4 | 29.5 | 29.8 | 29.6 | 29.6 | 30.0 |
| | 5 | 29.5 | 28.3 | 29.0 | 28.4 | 29.5 |
| 6 | 1 | 30.7 | 29.7 | 29.5 | 31.0 | 30.9 |
| | 2 | 31.1 | 29.8 | 28.9 | 28.9 | 29.4 |
| | 3 | 33.3 | 32.2 | 31.5 | 31.8 | 31.2 |
| | 4 | 28.5 | 29.3 | 29.0 | 29.3 | 29.6 |
| | 5 | 28.6 | 28.5 | 28.2 | 28.4 | 28.8 |

### Termination

On the scheduled day of termination, all designated animals were euthanized by cervical dislocation.

### Necropsy

At termination of observation period, animals were sacrificed and investigated macroscopically for gross necropsy and changes. Designated animals were necropsied soon after the time of death. A full, detailed gross necropsy that included careful assessment of the external surface of the body, all orifices, thoracic, and abdominal cavities, and contents within each cavity was performed. No treatment-related changes were observed. A summary of macroscopic observations is in Table 7.

TABLE 7
Summary of Macroscopic Observations

| | Group | | | |
|---|---|---|---|---|
| | Comb. 1 | Comb. 4 | Comb. 5 | Comb. 6 |
| | (2000 | (2000 | (2000 | (2000 |
| | mg/kg) | mg/kg) | mg/kg) | mg/kg) |
| Number of Animals | 5 | 5 | 5 | 5 |
| Observed | | | | |
| No abnormalities | 5 | 5 | 5 | 5 |
| detected | | | | |
| Abnormality detected | 0 | 0 | 0 | 0 |

### Data Evaluation and Statistical Analysis

All data are interpreted in arithmetic means presented with standard error of mean (SEM).

A similar toxicity study was done for Combination 10. In a sighting study, doses were selected from the fixed dose levels of 5, 50, 300 and 2000 mg/kg, as recommended by the OECD Guideline for Testing of Chemicals Sighting study (Limit test) to allow for selection of the appropriate starting dose for the main study. The test substance was administered to single animals at the doses listed. Animals were observed closely for the first 24 hours, and kept for observation for 14 days. Parameters considered included response to external stimuli, grooming, general activity and movement. All animals were considered normal and did not express any abnormal behaviors or signs. No compound-related mortality or signs of toxicity were observed. A summary of the results is shown in Table 8.

TABLE 8
Summary of Sighting Study Observation

| | 5 | 50 | 300 | 2000 |
|---|---|---|---|---|
| Cage-side Observations | mg/kg | mg/kg | mg/kg | mg/kg |
| Number of Animals Observed | 1 | 1 | 1 | 1 |
| No abnormalities detected | 1 | 1 | 1 | 1 |
| Mortalities | 0 | 0 | 0 | 0 |

After determining the appropriate starting dose level from the limit test (sighting), specifically 2000 mg/kg, a total of five female mice were used to investigate the effect of Combination 10 in the main study. Animals were observed closely for the first 24 hours, and kept for observation for 14 days. Cage-side observations are shown in Table 9. Parameters considered included response to external stimuli, grooming, general activity and movement. No treatment-related signs were reported. A summary of detailed clinical observations is also in Table 9. Parameters considered included passiveness to capturing, skin texture, respiratory sounds, abnormal discharges, and abnormal abdominal or urogenital presentation. All animals were handled and observed. Animals of treated with Combination 10 at 2000 mg/kg showed normal response to capture. No treatment-related observations were observed. No compound-related body weight changes were noted. No significant differences were noted in absolute body weight or total body weight changes over the course of the study. No mortalities were reported.

TABLE 9

| Parameters Considered | Combination 1 (2000 mg/kg) |
|---|---|
| No abnormalities detected | 5 |
| Abnormality detected | 0 |
| Response to External Stimuli | 0 |
| General activity (less) | 0 |
| Grooming | 0 |
| Sluggish Movement* | 0 |
| Detailed Clinical Observations | |
| Number of Animals Observed | 5 |
| No abnormalities detected | 5 |
| Abnormality detected | 0 |

*less activity was noted only during first hour from dosing

On the scheduled day of termination, all designated animals were euthanized by cervical dislocation. Designated animals were necropsied soon after the time of death for gross necropsy and changes. A full, detailed gross necropsy that included careful assessment of the external surface of the body, all orifices, thoracic, and abdominal cavities, and contents within each cavity was performed. No treatment-related changes or abnormalities were determined.

### In Vitro Cytotoxicity of Combination 1 or Combination 10 in Vero-E6 Cells

Ten (10) mg of Combination 1 (WA1) or Combination 10 (WA10) was dissolved in 1 ml of 20% ethanol:80% water. The suspended compounds were centrifuged briefly to remove the debris and the supernatants were transferred into new tubes. Eight serial one-half log₁₀ dilutions of Test Articles (5000, 1580, 500, 158, 50, 15.8, 5, and 1.58 μg/mL) plus no-compound control were prepared in Dulbecco's Modified Eagle Medium (DMEM). The same eight serial dilutions of 20% ethanol: 80% water solution without test articles were prepared in DMEM as a diluent control with final concentrations of 10%, 3.16%, 1.0%. 0.316%, 0.1%, 0.01%, and 0.00316% Ethanol. Supernatants from 96-well tissue culture plates containing monolayers of Vero-E6 cells seeded at a density of ˜5×10⁴ cells per well and incubated at 37° C. and 5% CO₂ for 24 hours were removed and replaced with 100 μl of each dilution of WA1, WA10, ethanol:water solution control, or medium only, in triplicate. The plates were incubated at 37° C. and 50% CO₂ for 24 hours incubation. Following incubation, the cell culture medium was removed and cell viability was assessed using Cell Titer-Glo® Luminescent Cell Viability Assay per manufacturer's instructions (Promega, G7572, Madison, WI, USA). Luciferase activity was measured in a GLoMax Plate Reader. Data were imported into GraphPad prism, normalized and evaluated by non-linear regression after adjusting normalized values for toxicity of ethanol diluent.

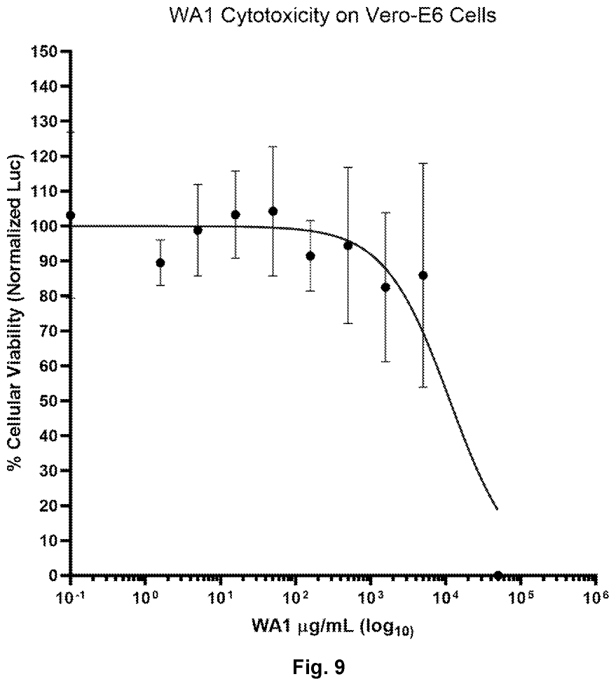

The dose response curves for Combination 1 and Combination 10 are presented in FIGS. 9 and 10, respectively, and the results of non-linear regression analysis are shown in Table 10.

TABLE 10
Log(inhibitor) vs. Normalized Response

| | WA-10 | WA-1 |
|---|---|---|
| Best-fit values | | |
| LogCC50 (μg/mL) | 3.897 | 4.056 |
| CC50 (μg/mL) | 7894 | 11375 |
| 95% C.I. (profile likelihood) | | |
| LogCC50 (μg/mL) | 3.653 to 4.160 | 3.782 to 4.343 |
| CC50 (μg/mL) | 4502 to 14439 | 6060 to 22022 |
| Qulatity of fit | | |
| Degrees of Freedom | 19 | 29 |
| R squared | 0.8472 | 0.7153 |
| Sum of squares | 3129 | 9263 |
| Sy.x | 12.83 | 17.87 |
| Number of points | | |
| Number of X values | 30 | 30 |
| Number of Y values analyzed | 20 | 30 |

### Cytokine Inhibition Study

### Objective of the Study

This study was designed to test if combinations described herein can reduce proinflammatory cytokine storm that may occur during COVID-19 infection or in any immune-mediated disease. In the following experiments, we have used lipopolysaccharide (LPS) to induce cytokine storm in an animal model of SEPSIS.

### Methods

To establish the maximum tolerated dose of LPS to induce endotoxemia symptoms but without causing deaths, a scale-up/down technique was used. LPS was injected intraperitoneally (i.p.) into C57Bl/6 female mice (Matalka et al. 2005; Farhana A, Khan 2021). Mice were observed closely during the first 8 hours and then kept under observation for three days post LPS administration. Following the up/down technique, it was found that 5 mg/kg of LPS dose was the lower best dose to show symptoms but without inducing deaths. The symptoms started within half an hour and were mostly seen within 4 to 4.5 hours, including inactivity, lethargy, hyperventilation, hunchback, and diarrhea.

### Dose Formulation

Lyophilized LPS (Chem-Cruz, Texas, USA) was reconstituted with endotoxin free phosphate buffered saline (PBS) (GE Life Science, Boston, USA) to form 10 mg/ml concentration and a volume of 1.0 ml. Reconstituted LPS was aliquoted into 100 μl and stored at −20° C. Doses of LPS were prepared for a sighting study which will provide the nonlethal tolerated dose of LPS to be employed at the main study.

All Test Articles (TAs), Combinations 1 and 10, were stored in their original package as received. All preparations were freshly dissolved in 0.5% medium viscosity carboxymethyl cellulose (CMC) (Sigma-Aldrich, Missouri, USA) directly before experimentation or dosing and were discarded after use.

### Mice

Thirty-eight female C57Bl/6 mice (Taconic Co., USA) 8-10 weeks old were used for the study. The study was performed at the University of Petra Pharmaceutical Center Animal Facility, Amman-Jordan. Healthy non-pregnant mice were randomly selected, marked for individual identification, and kept in their cages for 8 days prior to the initiation of the study to allow acclimatization.

Study groups were assigned into polysulfone individually ventilated cages (IVC) system; Eurostandard Type II 1285L (floor area 542 cm², with wired inner fitting lid) (Techniplast, Italy). Mice were randomly assigned and housed with no more than 5 per cage, under controlled environmental conditions.

The animal housing room maintains a temperature of 22° C. (±3° C.) and 65% (±5) relative humidity and artificial photoperiodic lighting of 12 hour-light and 12 hour-dark cycle.

Mice were provided with standard rodent chow diet offered ad libitum. It is considered that there are no known contaminants in the feed that could interfere with the objectives of the study. Prior the administration of LPS doses, animals were fasted for 4 hours from food.

Mice were provided with clean drinking water ad libitum. It is considered that there are no known contaminants in the water that could interfere with the objectives of the study. Throughout the study (even under food fasting conditions) animals were provided with water ad libitum.

### Establishing Endotoxemia

To establish the maximum tolerated nonlethal dose of LPS to induce endotoxemia signs but without deaths, an intraperitoneal (i.p.) LPS administration was performed. The starting dose for the sighting study was 5 mg/kg followed by increasing or decreasing the doses stepwise, namely 10, 20 and 0.5, 1 mg/kg, sequentially. Animals were closely observed for the first 24 hours and kept for observation for 5 days. The purpose of the sighting study is to allow selection of the appropriate dose of LPS for the main study. The test substances were administered to 2 animals per dose level, in a sequential manner.

### Cytokine Modulation

After determining the appropriate LPS dose for inducing the in vivo cytokine model namely; 5 mg/kg, a total of five female mice were used to investigate the effect of Combinations 1 and 10 on the model. Animals were treated with the TAs prior to the induction of endotoxemia.

To determine efficacy of oral administration of (Combination 1 and 10) to regulate or modulate proinflammatory induced cytokines (TNF-α, IL-6, and IL-1β) following LPS administration in mice, the TAs were delivered in vivo twice a day of 100 mg/kg per single dose by oral gavage for 7 days using an oral gavage stainless steel needle of suitable size. On the scheduled day of terminal, mice had their final dose of TAs and 1 hour later LPS was administrated in i.p.

Four hours after LPS administration, all designated animals were euthanized using inhaled isoflurane (5% mixed with oxygen) followed by exsanguination. Designated animals were necropsied soon after the time of death. A full, detailed gross necropsy which included careful assessment of the external surface of the body, all orifices, thoracic, and abdominal cavities, and contents within each cavity was performed. Only organs requested were collected for further ingestion.

After scheduled euthanasia, potential target tissues blood, lungs, kidneys, and liver were weighed, placed in “Cytokine Extraction” buffer and homogenized. Tubes were then centrifuged, and supernatant were collected and stored at −80° C. for further analysis of target cytokines.

### Cytokines Measured

Cytokines IL-6 (M6000B), TNF-α (MTA00B), and IL-1β (MLB00C) were purchased from R&D systems and used according to the manufacturer's procedure. The data were analyzed using analysis of variance with Tukey as a post hoc test.

### Data Analysis

Data were analyzed using ANOVA with post hoc test will be used to test for significant difference between treated groups of each organ.

### Results

### Clinical Observations During Seven Days of TAs Administration Prior to LPS

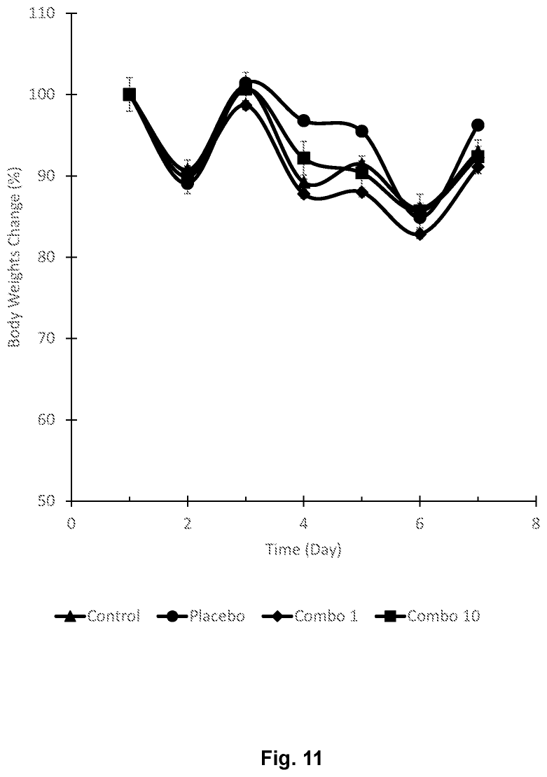

FIG. 11 shows that no treatment-related changes in weight were noted after seven days of either combination. No significant differences were noted in absolute body weight or total body weight changes over the course of the study.

Food consumption during day 1 was considered comparable to normal. However, consumption of food at day 7 was found higher than normal, suggesting an increase in appetite. Likewise, water consumption was comparable to normal at day 1 and an increase in water consumption was noted at day 7 from treatment. Collectively, concluding increase in appetite and thirst in animals after treatment with Combo 1 and 10.

Percent change in stool output was considered comparably similar to normal. In line with readings of water consumption, a direct increase in urine output was noticed. We concluded that no treatment-related changes in urine and stool output were considered clinically significant.

At termination of observation period: Animals were sacrificed and investigated macroscopically for gross necropsy and changes. No treatment-related changes were concluded.

### Clinical Observations Following LPS Administration

Parameters for endotoxemia considered were passiveness to capturing, skin texture, respiratory sounds, abnormal discharges, and abnormal abdominal or urogenital presentation. During treatment with TAs, mice showed normal behavior in comparison with the control and placebo groups. After induction of the in vivo endotoxemia model, LPS-treated groups showed calmness and passiveness upon capturing and skin condition was poor in terms of grooming. Animals showed abnormal diarrhea and difficulty in breathing (apnea). Despite presence of the signs in groups treated with Combo 1 and 10, signs were more pronounced in the Placebo group. No treatment-related mortality or signs of toxicity were noted to be clinically significant.

### Cytokine Analysis Following TAs and LPS Administration

FIG. 12 shows aspects of the effect of pre-administration of Combinations 1 and 10 for seven days at 200 mg/kg/day on LPS-induced IL-6 production in blood, kidneys, and lungs. There is a pattern of reduction of IL-6 production and most notably in the lungs. Error bars represent standard error of the mean. (***p<0.001, **p<0.01, and *p<0.05).

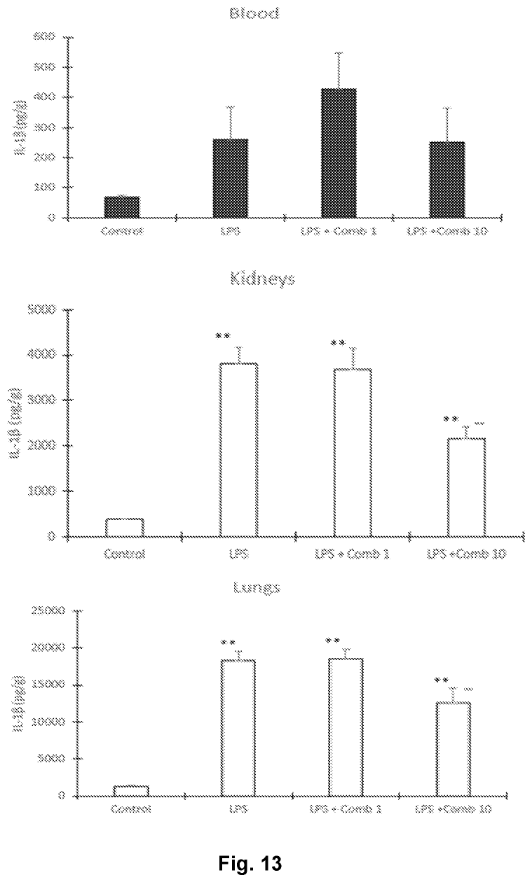

FIG. 13 shows aspects of the effect of pre-administration of Combinations 1 and 10 for seven days at 200 mg/kg/day on LPS-induced IL-1β production in blood, kidneys, and lungs. Combination 10 reduced LPS-induced IL-1β production in the lungs and kidneys. Error bars represent standard error of the mean. (***p<0.001, ** or --p<0.01, and * or -p<0.05).

Following LPS administration, IL-6, IL-1β, and TNF-α levels significantly increased in the blood, lungs, and kidneys. However, oral pre-administration of the two Combinations 1 and 10 (200 mg/kg/day) reduced LPS-induced cytokine increase with a specific pattern. Oral administration of combination 10 reduced IL-1β and IL-6 levels in the lungs and kidneys by 32% (p<0.05), 40% (p<0.01), and 31 and 32%, respectively (FIGS. 19 and 20). On the other hand, combination 1 administration showed a trend of decreasing LPS-induced IL-6 levels in the lungs, kidneys, and blood but without reaching significance (FIGS. 12 and 13). Although LPS-induced modest TNF-α levels in the organs tested, these levels were not indicative of endotoxemia and, therefore, not of clinical importance to be discussed.

### CONCLUSION

The data from the cytokine study showed that Combination 10 at 200 mg/kg/day for seven days reduced LPS-induced IL-6 and IL-1β in lungs and kidneys, whereas combination 1 showed some trend of reduction. The main reason in both combination responses is the presence of Anacyclus pyrethrum in combination 1, which contains polysaccharides from its aqueous extracts that has been shown to exhibit immunostimulant activities. On the other hand, combination 10 contains Elettaria cardamomum that has immunomodulatory compounds such as cardamonin. Cardamonin was found to regulate a transcription factor, nuclear factor-κB (NF-κB), which enhances the transcription of proinflammatory cytokines. Furthermore, hesperidin, safranal, and crocin, which are present in the Manuka Honey and Crocus sativus extracts, respectively, were found to regulate proinflammatory cytokines. Other studies combined with the present data confirm that combination 10 contains plant extracts that regulate proinflammatory cytokines. Such cytokine regulation can help in SARS-CoV-2 infection.

### Initial Clinical Trials

Initial clinical trials have been conducted with a small group of participants and plans are underway for large trials to begin. While no active research on humans has been done to date on other related viruses, given the compositions, we plan to start trials on the viability of this drug to treat other members of the SARS virus family and potentially HIV.

The prepared extract can be in a powder form or in a volatile oil. The composition can also be administered parenterally. In limited trials on humans, the mixture as a powder mixed with water resulted in significant improvement to patients spanning multiple age groups with no side effects, including the over-60 group who are the highest risk group from COVID-19.

### First Trial

### Methods

Seven patients with COVID-19 symptoms were dosed with the extracts orally. The patient information is as follows.

| | |
|---|---|
| Female | 64 years old |
| Female | 28 years old |
| Female | 27 years old |
| Female | 34 years old |
| Female | 24 years old |
| Female | 25 years old |
| Female | 45 years old |

### Preparation and Dosage

In the trial, the composition given to each patient was prepared from the following list of herbal components as follows:

| | |
|---|---|
| 10 grams of Cardamom seed | 10 grams of dried saffron |
| 10 grams of dried hyoscamus | 10 grams of dried Valeriana |
| leaves | wallichi |
| 10 grams of dried | 10 grams of dried Helleborus |
| Anacyclus pyrethrum roots | Vesicanus |
| 20 grams of dried Euphorbia | |

The ingredients were mixed together, then crushed and ground using mortar and pestle. The mixture was sieved and then mixed with non-foamed Manuka honey, making sure no bubbles were formed. A single serving is only 378 mg of this mixture. Each single serving was added to 1 cup of sterile water (warm or hot to dissolve the mixture). Five grams of dried (aerial parts) sweet violet was added to the mixture. Each patient was given a single serving size of the mixture above once daily, at nighttime for three days in a row. The serving was given at night, because the composition has anesthetic-like side effects that help the patient relax and sleep, which aids with the recovery. Also, it was recommended that the patient can continue taking only the sweet violet (in a cup of sterile water) once daily for five days to a week, to eliminate symptoms such as cough and runny nose.

All patients had their symptoms reduced within a 24-hour period. The 64-year-old female patient with the most severe symptoms, had relief within an hour, especially the chest pain and difficulty breathing.

### Second Trial

### Methods

### Preparation and Dosage

In this trial, the composition given to each patient was prepared from the following list of herbal components as follows: 5 grams of cardamom seed, 5 grams of dried saffron, without or with 5 grams of dried Anacyclus pyrethrum roots (some patients did not receive Anacyclus extract, see tables for more details).

The ingredients were mixed, then crushed and ground using mortar and pestle. The mixture was sieved and then mixed with non-foamed Manuka honey, making sure no bubbles were formed. Then eight grams of dried sweet violet (aerial parts) were added to the mixture. The mixture was then sieved when the aerial parts of the sweet violet color was faded.

Every single serving was 0.6-0.7 mg of this mixture added to 1 cup of sterile water.

The Ficus carica extract was prepared by adding three to six figs to 1 liter of hot water and left for at least 6 hours. Each patient drank 1 cup from the first mixture (Manuka, saffron, cardamom, sweet violet, with or without Anacyclus) and another cup from the figs extract every 10-12 hours (i.e., twice daily) for five days.

*Note: The two children were given 1:4 (quarter) the dosage given to adults, according to their weight.

Treatment Group: Eleven positive unvaccinated COVID-19 patients with the age range 25-74 years old (5 males and 6 females) were accepted to enroll in the study. Before administrating the herbal product, all patients had symptoms of COVID-19 such as fever, fatigue, sour throat, cough, loss of smell/taste, and headache. The characteristics of the are shown in Table 11. After three days of administration of Combination 10 alone, or Combination 10 with Anacyclus pyrethrum, symptoms improved in all patients (Table 11). In 3 days, the two patients with shortness of breath became and other symptoms became symptomless, except for fatigue in one patient. After three-day treatment, 27.3% of patients had no symptoms, and the symptoms in 72.7% of patients were much less except for fatigue and loss of smell. A few days later, 9 out of 11 patients (2 did not do the test) had a negative PCR for COVID-19 (Table 11).

Preventive Group: Fourteen subjects were in close contact with positive PCR COVID-19 patients accepted to take WAHI's herbal combination as a prophylactic. These patients did not test for COVID-19 and were given the herbal product on the same day or 24 hours of possible exposure. The characteristics of the unvaccinated COVID-19 subjects who were in direct contact with a positive PCR patient and were not wearing masks for 1 hour period are shown in Table 12. Subjects were administered Combination 10 (without or with Anacyclus pyrethrum) for 5 days as shown in Table 12. Thirty-six percent (36%) of the subjects did not develop any COVID-19 symptoms. Nine patients (64%) developed COVID-19-like symptoms such as fever, fatigue, cough, sour throat, headache, and loss of smell/taste. After three days of treatment with Combination 10 alone, or Combination 10 with Anacyclus pyrethrum, 56% of patients' symptoms cleared, and 44% of patients' symptoms became much less significant.

TABLE 11

| | | | | | | Symptoms | | | |
|---|---|---|---|---|---|---|---|---|---|
| | | | | | | after | | | |
| Patient | | | | Medication Given with | Chest X- | 3 days of | | | Combination |
| Number | Age | Gender | Symptoms | WAVBS | RAY scan | WAVBS* | Notes | Recovery | Used |
| 1 | 74 | male | Shortness of | Omeprazole 40 mg, | ND | fatigue | The 3 days course of | Negative | Combo 10 |
| | | | breath, fever, loss | Vitamin C 1000 mg, Zinc | | | Azithromycin was | | |
| | | | of smell and taste | 10 mg, Azithromycin 6 | | | swallowed in 24 hours, | | |
| | | | sensation, fatigue | days course, Ambroxol, | | | Gave a Negative PCR | | |
| | | | and pain in joints | Vitamin D 50000 IU once | | | test before the 10 Days | | |
| | | | | per week, Paracetamol | | | period and in the 14 | | |
| | | | | 500 mg X2 every 8 hours if | | | day. smoked once | | |
| | | | | necessary | | | | | |
| 2 | 67 | male | Shortness of | Zinc 20 mg. Azithromycin | ND | No | Started smoking after | Negative | Combo 10 |
| | | | breath, difficulty in | (3 days course), Vitamin C | | symptoms | 14 days from the day of | | with |
| | | | breathing, fever, | 1000 mg, Vitamin D | | | infection. | | Anacyclus |
| | | | diarrhea, headache, | 50000 IU per week and | | | | | pyrethrum |
| | | | fatigue, loss of | Paracetamol 500 mg X2 | | | | | |
| | | | smell and taste | every 8 hours | | | | | |
| | | | sensation and pain | | | | | | |
| | | | in joints | | | | | | |
| 3 | 69 | female | fever, headache | same Medication Taken | no | Fatigue | Reported Negative on | Negative | Combo 10 |
| | | | and fatigue | before and took a one 3 | Inflammation | | the 14th day | | |
| | | | | days course of | | | | | |
| | | | | Azithromycin | | | | | |
| 4 | 29 | male | Fever, headache, | Vitamin C 1000 mg, Zinc | ND | No | Started smoking 5 days | Negative | Combo 10 |
| | | | sore throat, fainting | 15 mg, Vitamin D 50000 | | symptoms | after infection, Done a | | with |
| | | | | IU per week | | | PCR test on the 4th, | | Anacyclus |
| | | | | | | | 8th, 10th and 14th all | | pyrethrum |
| | | | | | | | were positive, but on | | |
| | | | | | | | the 16 it was Negative | | |
| 5 | 19 | male | fever, fatigue | Vitamin C 1000 mg, Zinc | ND | No | | ND | Combo 10 |
| | | | | 15 mg, Vitamin D 50000 | | symptoms | | | with |
| | | | | IU per week | | | | | Anacyclus |
| | | | | | | | | | pyrethrum |
| 6 | 39 | female | fever, fatigue, sore | Omeprazole 40 mg, | ND | Fatigue and | | Negative | Combo 10 |
| | | | throat, loss of | Vitamin C 1000 mg, Zinc | | headache, | | | with |
| | | | smell and taste | 10 mg, Azithromycin 6 | | loss of | | | Anacyclus |
| | | | sensation, | days course, Ambroxol, | | smell | | | pyrethrum |
| | | | headache, pain in | Vitamin D 50000 IU | | and taste | | | |
| | | | the joints and dry | ONCE IN THE 14 DAYS, | | sensation | | | |
| | | | cough | Paracetamol 500 mg X2 | | | | | |
| | | | | every 8 hours if necessary | | | | | |
| 7 | 36 | male | Fever, Fatigue and | Augmentin 500 mg for a | ND | Improvements | No improvement in | Negative | Combo 10 |
| | | | loss of smell and | week, Vitamin C 1000 mg, | | in smell | smell and taste | | with |
| | | | taste sensation | Ambroxol, Zinc 10 mg | | and taste | sensation | | Anacyclus |
| | | | | | | sensation | | | pyrethrum |
| 8 | 52 | female | Fever, fatigue, Dry | No Medication | ND | Fatigue | | ND | Combo 10 |
| | | | cough | | | | | | |
| 9 | 34 | female | Fever, fatigue, sore | Zinc 20 mg. Azithromycin | ND | Fatigue, | | Negative | Combo 10 |
| | | | throat, loss of | (3 days course), Vitamin C | | Loss of | | | with |
| | | | smell and taste | 1000 mg, Omeprazole | | smell | | | Anacyclus |
| | | | sensation, | 20 mg and Paracetamol | | | | | pyrethrum |
| | | | headache, pain in | 500 mg X2 every 8 hours | | | | | |
| | | | the joints and dry | | | | | | |
| | | | cough | | | | | | |
| 10 | 25 | female | Fever, fatigue, sore | Zinc 20 mg. Azithromycin | ND | Fatigue, | | Negative | Combo 10 |
| | | | throat, loss of | (3 days course), Vitamin C | | Loss of | | | with |
| | | | smell and taste | 1000 mg, Omeprazole | | smell | | | Anacyclus |
| | | | sensation, | 20 mg and Paracetamol | | | | | pyrethrum |
| | | | headache, pain in | 500 mg X2 every 8 hours | | | | | |
| | | | the joints and dry | | | | | | |
| | | | cough | | | | | | |
| 11 | 37 | female | Fever, Fatigue, | No Medication | ND | Fatigue, | | Negative | Combo 10 |
| | | | Headache | | | Loss of | | | |
| | | | | | | smell | | | |

TABLE 12

| | | | | | Symptoms | | | |
|---|---|---|---|---|---|---|---|---|
| | | | | | after | | | |
| | | | | | 3 days of | | | |
| Patient | | | | Medication Given with | WAVBS* | | | Combination |
| number | Age | Gender | Symptoms | WAVBS | given | Notes | Recovery | Used |
| 1 | 67 | female | Dry cough, loss of smell | Vitamin C 1000 mg, | no | Continued smoking | Not | Combo 10 |
| | | | sensation, fever, fatigue, | Paracetamol 500X2 Every 8 | symptoms | during 14 days | Determined | |
| | | | diarrhea, headache, sore | hours If necessary | | | | |
| | | | throat and pain in joints | | | | | |
| 2 | 34 | female | Fatigue, fever and headache | No medication | fatigue | stopped smoking | ND | Combo 10 |
| | | | | | | during the first 3 | | |
| | | | | | | days | | |
| 3 | 28 | female | fever, loss of smell and | Zinc 20 mg. Azithromycin (3 | Dry cough, | The smelling | ND | Combo 10 |
| | | | taste sensation, sore throat, | days course), Vitamin C 1000 | loss of | sensation returned | | with |
| | | | headache, fatigue and | mg, Vitamin D 50000 IU per | smell | back to normal after | | Anacyclus |
| | | | shortness of breath | week, Dry cough syrup, | and taste | one more from the | | pyrethrum |
| | | | | Clarinase (psuedoephedrine/ | sensation, | day of infection. | | |
| | | | | Loratidine) | fatigue, | (continued smoking | | |
| | | | | | runny nose | during infection) | | |
| 4 | 27 | female | fever, loss of smell and | Vitamin C1000 mg | Fatigue, | stopped smoking | ND | Combo 10 |
| | | | taste sensation, sore throat, | | runny nose, | | | with |
| | | | headache, fatigue, shortness | | dry cough | | | Anacyclus |
| | | | of breath, dry cough, runny | | and | | | pyrethrum |
| | | | nose | | improvement | | | |
| | | | | | in smell | | | |
| | | | | | and taste | | | |
| 5 | 25 | female | fever, loss of smell and | No medication | Dry cough, | Continued smoking | | Combo 10 |
| | | | taste sensation, sore throat, | | fatigue, | during 14 days | | with |
| | | | headache, fatigue and Dry | | runny | | | Anacyclus |
| | | | cough | | nose | | | pyrethrum |
| 6 | 28 | female | fever, fatigue, sore throat, | Vitamin C 1000 mg, Zinc | No | stopped smoking in | Negative | Combo 10 |
| | | | loss of smell and taste | 15 mg, Vitamin D 50000 IU | symptoms | the first two days | | with |
| | | | sensation, headache | per week | | | | Anacyclus |
| | | | | | | | | pyrethrum |
| 7 | 30 | female | no symptoms | No medication | No | Continued smoking, | ND | Combo 10 |
| | | | | | symptoms | Took WAVBS as a | | with |
| | | | | | | prophylaxis | | Anacyclus |
| | | | | | | | | pyrethrum |
| 8 | 2 | male | fever | No medication | No | Took WAVBS as a | ND | Combo 10 |
| | | | | | symptoms | Prophylaxis | | |
| 9 | 3 | male | no symptoms | No medication | No | Took WAVBS as a | ND | Combo 10 |
| | | | | | symptoms | Prophylaxis | | |
| 10 | 39 | female | fever, fatigue, headache, | Vitamin C 1000 mg | No | Took WAVBS as a | ND | Combo 10 |
| | | | sore throat, loss of smell | | symptoms | Prophylaxis | | with |
| | | | and taste sensation, | | | | | Anacyclus |
| | | | | | | | | pyrethrum |
| 11 | 42 | male | no Symptoms | No medication | No | Took WAVBS as a | ND | Combo 10 |
| | | | | | symptoms | Prophylaxis | | with |
| | | | | | | | | Anacyclus |
| | | | | | | | | pyrethrum |
| 12 | 47 | female | no symptoms | No medication | No | Took WAVBS as a | ND | Combo 10 |
| | | | | | symptoms | Prophylaxis | | |
| 13 | 45 | Female | No symptoms | No medication | No | Took WAVBS as a | Negative | Combo 10 |
| | | | | | symptoms | Prophylaxis | | with |
| | | | | | | | | Anacyclus |
| | | | | | | | | pyrethrum |
| 14 | 41 | Male | Fatigue, fever, Headache | Zinc 20 mg. Paracetamol | No | Took WAVBS as a | ND | Combo 10 |
| | | | | 1000 mg when needed | symptoms | Prophylaxis | | |
| | | | | maximum twice a day, | | | | |
| | | | | Vitamin C 1000 mg, Vitamin | | | | |
| | | | | D 1000 IU | | | | |

*WAVBs: WAHI ANTI-viral botanical solution

### In Vivo Testing in a Murine Model of SARS-CoV-2 Infection

### Study Design

The ability of compositions described herein, such as Combination 10, to inhibit the replication of SARS-CoV-2 and protect transgenic mice from COVID-19 disease can be determined in a murine model of SAR-CoV-2 as described below.

The studies are performed in 40 transgenic female k18-hACE2 mice (Jackson Labs) aged 6-8 weeks (at arrival) expressing human ACE-2 receptor.

Cage-matched mice in groups of fifteen mice each (for virally treated groups) and ten for uninfected and untreated group (5 mice per cage) are procured and acclimated for 7 days prior to study initiation. Mice are lightly anesthetized with isoflurane inhalation and treated twice daily by oral gavage for 7 days with test article or vehicle control prior to and each day following challenge with a pre-titrated lethal dose of SARS-CoV-2 (Italian) by nasal instillation. One group of 10 mice serves as an untreated uninfected control group (Table 13).

Mice are evaluated daily for general health during the acclimation period and prior to viral challenge. Clinical observations, body weight, and body temperatures are recorded daily following viral challenge.

Five (5) mice in each group are humanely euthanized three (3) days post-viral challenge (Study Day 10) and viral titers in the lung tissues determined by TCID₅₀ analysis. A portion of the lung tissue is collected from euthanized mice and placed into 10% formalin for histopathology.

The remaining mice (10 mice for the treated groups, and 5 mice for the uninfected and untreated) are observed for mortality, clinical signs of illness, body weight, and body temperature through Study Day 21 (14 days post virus challenge). Lung tissue is collected for determination of viral titers and histology from mice euthanized in extremis (due to qualifying health scores). Mice surviving until Study Day 21 (14 days post challenge) are humanely euthanized and lung tissues are collected for histopathology and determination of the presence of viral RNA by qRT-PCR.

TABLE 13
Study groups, treatments, and end-point analysis

| | | Dose Route/vol* | Virus challenge | | |
|---|---|---|---|---|---|
| | | Frequency | Route/vol. | Clinical Obs | |
| Group (n) | Test article | (Days 0-21) | (Day 7) | (Days 7 to 21) | End point analysis |
| 1 (10) | Vehicle | Oral Gavage/<1 mL | None | Daily | TCID₅₀ and |
| 2 (15) | Vehicle | 2X daily Days 0 to 21 | SARS-CoV-2 | Clinical score | histology in lung |
| | | | (ITN) IN/50 μL | Body weight | tissue 5 mice each |
| 3 (15) | Combination | | SARS-CoV-2 | Body temp. | group Day 10. |
| | 10 | | (ITN) IN/50 μL | | TCID₅₀ and |
| | | | | | histology in lung |
| | | | | | tissue of mice |
| | | | | | euthanized in |
| | | | | | extremis. |
| | | | | | Viral RNA and |
| | | | | | histology of lung |
| | | | | | tissue in mice |
| | | | | | survived 21 Days |

*Dose volumes for test article and vehicle control is dependent on test article concentration and dose.

### Follow-Up Points

Animals are monitored for viability, scored for clinical signs of illness, body temperature, and weighed daily following viral challenge for up to 14 days post challenge.

Five mice from each group are euthanized on Day 10 (3 days post viral challenge) for determination of viral titers (TCID₅₀) in lung tissue and histopathology of lung tissue.

Viral titers (TCID₅₀) in lung tissue and histopathology of lung tissue for mice euthanized in extremis.

Mice surviving for 21 days (14 days post challenge) are humanely euthanized and lung tissues are collected for histopathology and determination of the presence of viral RNA by qRT-PCR.

Post-challenge data reported for groups and individual animals include mortality, body weight, body temperature, and clinical scores for viral titers in lungs and lung histopathology on Day 10 (3 days post-challenge)-5 mice each group and on mice euthanized in extremis are recorded. Viral RNA in lungs and lung histopathology on mice surviving for 21 days (14 days post challenge) are also recorded.

### Formulations

The embodiments of the compositions containing plant extracts described in this disclosure may be formulated with one or more pharmaceutically acceptable additives. The additive may be a carrier, an excipient, a binder, a colorant, a flavoring agent, a preservative, a buffer, a diluent or dilutant, and/or combinations thereof. Other pharmaceutically acceptable additives include sterile liquids, such as water and oils, including those of petroleum, animal, vegetable, or synthetic origin, such as peanut oil, soybean oil, mineral oil, sesame oil, or similar oils, water or saline aqueous solutions, aqueous dextrose and glycerol solutions. The pharmaceutically acceptable additive useful for formulating a dosage form comprising the compositions of this disclosure will depend, among other factors, on the elected administration route.

The compositions may be formulated in a dosage form selected from the group consisting of a capsule, a cachet, a pill, a tablet, a powder, a granule, a pellet, a bead, a particle, a troche, a lozenge, and a gel.

The compositions may be in solid or liquid form for oral administration. Solid forms include tablets, caplets, pill, capsules, gelcaps, powders, granules, gummies and the like. A tablet or pill is usually a compressed preparation that may contain, for example but not limitation, 5-10% of the active extract composition; 80% of fillers, disintegrants, lubricants, glidants, and/or binders; and 10% of compounds that provide easy disintegration, disaggregation, and dissolution of the tablet in the stomach or the intestine. The dissolution time can be modified for a rapid effect or for sustained release. Special coatings can make the tablet resistant to the stomach acids such that it only disintegrates in the duodenum, jejunum and/or colon as a result of enzymatic action or alkaline pH. Pills can be coated with sugar, varnish, or wax to disguise the taste.

A caplet is a compressed mixture of ingredients, similar to a tablet, which is formed into a capsule shape. It often has a film or gelatin coating to mask the taste and make it easier to swallow. Capsules generally comprise hard shells that can contain powders or granules, or liquids in some embodiments. A soft gel capsule or gelcap comprises a thicker, softer shell that may be easier to swallow. Gelcaps often comprise liquid or paste compositions inside the gelatin shell.

Gummies are chewable formulations comprising the active ingredients in a chewable gelatin base. They may come in a variety of flavors, colors, and shapes and may be desirable for subjects that may not like swallowing pills or capsules.

In some embodiments, the composition may be in the form of a biodegradable capsule containing the composition. In one embodiment, the composition is in the form of a supplement. In another embodiment, it is in the form of a pharmaceutical composition. In another embodiment, the compositions may be formulated with liposomes, polymeric micelles, microspheres or nanoparticles.

In some embodiments, such as for a nutritional supplement, the composition can be formulated as a powder that can be added to a liquid, for example a beverage, to prepare a drinkable solution or suspension of the combination. Liquid formulations may be formulated for oral, topical or percutaneous applications.

The formulated compositions may be manufactured according to conventional methods known by the skilled person in the art. For example, the compositions may be prepared using standard methods such as those described or referred to in the US Pharmacopoeias and similar reference texts.

In particular, the compositions of the present invention can be administered as a pharmaceutical composition or an over-the-counter therapeutic supplement, either of which comprises the compositions described herein and one or more pharmaceutically acceptable additives.

The compositions for use according to the invention may be administered as the sole active ingredient or in combination with other active ingredients. In a particular embodiment, the compounds are used as the sole active ingredient. In another particular embodiment, the compounds are used in combination with other active ingredients, such as vitamins.

Any of these forms may be formulated to provide a unit dose of the combination. A unit dose comprises the amount of a medication administered to an individual in a single dose. The unit dose may be 350-400 mg active ingredient mixture per capsule. The dose for children above 12 is 1-2 capsules per day. The dose for adults above 18 years is 2-4 capsules after major meals. Optionally, the unit dose may further comprise other constituents such as vitamin C, vitamin D and/or vitamin B₁₂. The unit dose may comprise 1000-2000 mg of vitamin C. The unit dose may comprise 1000 International Units (IU) of vitamin D3.

In the disclosed compositions, the botanical extract can be taken orally, to reduce side effects and maximize the benefits, with a maximum dosage of 5 grams per day. The sweet violet extract can be used to overcome the hepatotoxicity of other plants and reduce the viral symptoms, whereas the other plant extracts are used for antiviral, immunomodulatory and anti-inflammatory effects.

While the disclosure is described in conjunction with the enumerated aspects, it will be understood that they are not intended to limit the invention to those aspects. On the contrary, the invention is intended to cover all alternatives, modifications, and equivalents that may be included within the scope of the present invention as defined by the claims. One skilled in the art will recognize many methods and materials similar or equivalent to those described herein, which could be used in the practice of the present invention. The present invention is in no way limited to the methods and materials described. Unless otherwise defined, all technical and scientific terms used herein have the same meaning as commonly understood by one of ordinary skill in the art to which this disclosure belongs. All publications, patent applications, patents, and other references mentioned herein are incorporated by reference in their entirety.

1-42. (canceled)

43. A composition comprising (a) an extract of a plant belonging to the family Violaceae; (b) an extract of a plant belonging to the family Myrtaceae; and one or more additional plant extracts wherein the plants are selected from the Moraceae Zingiberaceae, Hyoscamus, Asteraceae, Euphorbiaceae, Iridaceae, or Valeriana families, wherein the composition exhibits suppression of a Coronavirus protease.

44. The composition of claim 43 comprising (a) an extract of a plant belonging to the family Violaceae; (b) an extract of a plant belonging to the family Mvrtaceae; and (c) an extract of a plant belonging to the family Moraceae.

45. The composition of claim 44 further comprising (d) an extract of a plant belonging to the family Iridaceae; and either
(e) an extract of a plant belonging to the family Asteraceae; or
(f) an extract of a plant belonging to the family Zingiberaceae.

46. The composition of claim 43 comprising extracts of plants belonging to the Violaceae, Myrtaceae, Zingiberaceae, Asteraceae, Iridaceae, and Valeriana families.

47. The composition of claim 43, further comprising one or more additional components selected from the group consisting of a carrier, an excipient, a binder, a colorant, a flavoring agent, a preservative, a buffer, a diluent, and any combination thereof.

48. The composition of claim 43 wherein the coronavirus is SARS-CoV-2 or variant thereof.

49. The composition of claim 48 wherein the viral protease is 3CL (Mpro) of SARS CoV-2.

50. The composition of claim 43 wherein the composition exhibits inhibition of coronavirus S1+S2 spike proteins binding to ACE2 in an individual exposed to the coronavirus.

51. The composition of claim 43, wherein the composition inhibits replication of the coronavirus in the individual exposed to the coronavirus.

52. The composition of claim 43 wherein the composition exhibits inhibition of transmembrane protease serine 2 enzyme of lung epithelial cells in the individual exposed to the coronavirus.

53. The composition of claim 43, wherein the composition inhibits a cytokine storm in the individual exposed to the coronavirus.

54. A method for treating coronavirus in an individual, the method comprising administering a therapeutically effective amount of a composition of claim 43 to the individual.

55. The method of claim 54, wherein the coronavirus is SARS-CoV-2 or variant thereof.

56. The method of claim 54, wherein the administration of the composition exhibits suppression of a coronavirus protease.

57. The method of claim 43, wherein the viral protease is 3CL (Mpro) of SARS-CoV-2.

58. The method of claim 43, wherein the composition inhibits replication of the coronavirus in the individual exposed to the coronavirus.

59. The method of claim 54 wherein the composition exhibits inhibition of coronavirus S1+S2 spike proteins binding to ACE2 in the individual exposed to the coronavirus.

60. The method of claim 54 wherein the composition exhibits inhibition of transmembrane protease serine 2 enzyme of lung epithelial cells in the individual exposed to the coronavirus.

61. The method of claim 54, wherein the composition inhibits a cytokine storm in the individual exposed to the coronavirus.

62. A method of preventing infection of an individual exposed to a coronavirus comprising administering a therapeutically effective amount of a composition of claim 43.
